(19) United States
(12) Patent Application Publication
(10) Pub. No.: US 2019/0336482 A1
(43) Pub. Date: Nov. 7, 2019

(54) COMPOSITIONS AND METHODS FOR TREATING AND DIAGNOSING OCULAR DISORDERS

(71) Applicant: TRANSLATUM MEDICUS, INC., TORONTO (CA)

(72) Inventor: Shelley Romayne BOYD, Toronto (CA)

(21) Appl. No.: 16/508,908

(22) Filed: Jul. 11, 2019

Related U.S. Application Data
(63) Continuation of application No. 15/580,766, filed on Dec. 8, 2017, now abandoned.
(60) Provisional application No. 62/190,550, filed on Jul. 9, 2015.
(60) Provisional application No. 62/180,432, filed on Jun. 16, 2015.

(51) Int. Cl. A61K 31/416; A61K 39/395; A61K 31/65; A61K 45/06; A61P 27/02; A61K 9/00; A61K 38/03; A61K 31/585; A61K 31/498; A61K 31/133; A61K 31/436

(52) U.S. Cl. CPC A61K 31/416; A61K 39/395; A61K 31/65; A61K 45/06; A61P 27/02; A61K 9/0048; A61K 2300/00; A61K 38/03; A61K 31/585; A61K 31/498; A61K 31/133; A61K 31/436; A61K 39/3955; A61K 9/0053

(57) ABSTRACT

Disclosed herein are methods, compounds, and compositions that are useful for the diagnosis, treatment, or prevention of an ocular disorder, including in the discovery of agents that are efficacious against these disorders.

### CROSS-REFERENCE TO RELATED APPLICATIONS

This application claims the benefit of U.S. Provisional Patent Application Nos. 62/180,432, filed Jun. 16, 2015 and 62/190,550, filed Jul. 9, 2015, the entire contents of which are herein incorporated by reference.

### FIELD OF THE INVENTION

This invention relates to, in part, methods and compositions that are useful for the diagnosis, treatment, or prevention of an ocular disorder, including the uses of immunomodulatory agents, that are efficacious against these disorders.

### BACKGROUND

Ocular disorders, which may reduce or eliminate ones sight, among other effects, are a major medical concern.

For instance, age-related macular degeneration (AMD) is a leading cause of ocular dysfunction, including irreversible blindness, especially in patients over 50 years old. All patients start with early so-called “dry” AMD. This is characterized by deposits that can be seen clinically in the posterior pole of the eye known as the macula. Advanced AMD can take two forms—late “wet” or late “dry”. The primary pharmaceutical option in advanced “wet” AMD is regular intra-ocular injections of antiangiogenic drugs. These injections are given after pathological new blood vessels grow beneath or into the central retina, where they leak, bleed or cause tissue damage and scarring. By contrast, there are no treatments for late or early dry AMD. Late dry AMD is characterized by the death or atrophy of the photoreceptors and their nurse cells, the retinal pigment epithelium (RPE). Patches of tissue loss are known as “geographic atrophy” GA.

Accordingly, there is presently a paucity of satisfactory agents for effective treatment of ocular disorders. Therefore, there remains a need for therapies that are useful for treating these disorders. Further, there is a need for effective diagnosis of these disorders and identification of patient populations that will respond to treatments of these disorders.

### SUMMARY OF THE INVENTION

Accordingly, in one aspect, the invention provides a method for treating or preventing an ocular disorder or its late blinding complication(s), comprising administering to a subject in need thereof an effective amount of an agent effective for the treatment of an ocular disorder, the agent optionally being an immunomodulatory agent (optionally selected from a Monocyte Chemoattractant Protein (MCP)-modulating agent, inclusive of MCP-1, MCP-2, and MCP-3)-modulating agents, including a compound of Formulae I-IV (as defined herein), PPAR gamma modulator, migration inhibitory factor (MIF) inhibitor, and chemokine receptor 2 (CCR2) inhibitor). In some embodiments, the immunomodulatory agent targets macrophages. In some embodiments, the immunomodulatory agent modulates M1/M2 polarization. In some embodiments, the immunomodulatory agent differentially modulates one or more of M1 and M2 macrophage activity in the subject. In some embodiments, the immunomodulatory agent modulates (e.g. reduces) M1 macrophage activity in the subject and/or modulates (e.g. increases or maintains) M2 macrophage activity in the subject. In some aspects, the invention relates to a method for treating or preventing an ocular disorder, comprising administering to a subject in need thereof an effective amount of an agent effective for the treatment of an ocular disorder, wherein the subject has an abnormal (e.g. increased or decreased, e.g. relative to an undiseased state) expression or activity of one or more of CD64, IDO, SOCS1, CXCL10 Marco, Nos2, II12b, Ptgs2 (Cox2), II23a (II23p19), Ido1, Adipoq, Ccl20, Socs3, Stat1, IL-6, and IL17 (subtype a). In some aspects, the invention relates to a method for treating or preventing an ocular disorder, comprising administering to a subject in need thereof an effective amount of an agent effective for the treatment of an ocular disorder, wherein the subject has an abnormal (e.g. increased or decreased, e.g. relative to an undiseased state) expression or activity of one or more of Ccl5, CD163, Cx3Cr1, FasIg, Gfap, Csf2, Icam1, Ifng, II10, II12b, II13, II17a, II18, II1b, II22, II4, II6, Klf4, Mrc1, Myd88, Nlrp3, Nos2, Ppary, Tgfb1, Tlr4, Tnf, Vcam1, MCP-1 (Ccl2), MCP-2 (Ccl5), MCP-3 (Ccl7), Ccr2, Socs1, Socs3, Stat1, Stat3, and Stat6. In one aspect, the present invention provides a method for treating or preventing an ocular disorder, comprising administering to a subject in need thereof an effective amount of a MCP-modulating agent. In some embodiments, the MCP-modulating agent targets the proximal, distal or other regulatory region of the MCP gene promoter/enhancer regions. In some embodiments, the present invention provides a method for treating or preventing an ocular disorder, comprising administering to a subject in need thereof an effective amount of a MCP-1 and/or MCP-2 and/or MCP-3-modulating agent.

In some aspects, the invention provides a method for treating or preventing an ocular disorder or its late blinding complication(s), comprising administering to a subject in need thereof an effective amount of an agent described in one or more of the following documents, the contents of which are hereby incorporated by reference in their entireties: International Patent Publication No. WO/2009/083436 (and U.S. Pat. Nos. 8,399,477 and 8,871,948); International Patent Publication No. WO/2009/109616 (and U.S. Pat. No. 8,461,194); International Patent Publication No. WO/2009/109618 (and U.S. Pat. Nos. 7,919,518 and 8,283,348); International Patent Publication No. WO/2009/109654 (and U.S. Pat. Nos. 8,314,099 and 8,569,297); International Patent Publication No. WO/2009/109613 (and U.S. Pat. No. 8,835,481); and International Patent Publication No. WO/2011/015501 (and U.S. Pat. No. 8,354,544).

In various aspects, the present methods are applicable to select subject populations. For example, the present methods, including embodiments in which the agent is a compound of Formulae I-IV as described herein, and the ocular disorder is dry AMD or Reticular Pseudodrusen (RPD) (also known as drusenoid deposits, reticular macular disease, etc.), pertain to a subject who is not undergoing treatment with or is undergoing treatment with and/or would benefit from treatment with and/or is unresponsive to one or more of an anti-factor D antibody (e.g. lampalizumab (Genentech)), an anti-β-amyloid (anti-AP) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE)). In various embodiments, including embodiments in which the agent is a compound of Formulae I-IV as described herein, and the ocular disorder is dry AMD or RPD, the subject may be classified by having one or more of the following: at least 1 druse greater than about 125 μm in diameter, one or more well-demarcated areas of GA lesions of a total area of about 2 to about 20 mm² in one or more eye, a best-corrected visual acuity score of greater than about 35 letters or a Snellen VA equivalent of about 20/200 or better, with or without evidence of prior or active choroidal neovascularization (CNV). In various embodiments, the subject may be classified by having one or more of the following: sub-retinal drusenoid deposits and/or reticular pseudodrusen and/or “diffuse trickling disease,” and/or other diseases of hyperfluorescent Fundus Autofluorescence (FAF) in the border or the junctional zone surrounding areas of GA. In various embodiments, the subject may be classified by having one or more of small round dots that can be multicentric to form target lesions, or inter-lacing ribbons as seen on en face imaging (e.g. as observed by white-light fundoscopy, blue-light, near-infra-red, infra-red, or multi-color imaging). Patients may have any one of more of these known diagnoses, or a disorder characterized by sub-retinal or intra-retinal fluid that may, in some embodiments, lead to distortion of the outer retina layers. Patients may be afflicted with any of the ocular disorders provided herein.

In various aspects, the present invention relates to the use of delayed near infrared analysis (DNIRA) as a surrogate biomarker for diagnosis and/or prognosis and/or progression of an ocular disorder (including, without limitation AMD and RPD). In various aspects, the present invention further relates to the use of DNIRA as entrance inclusion or exclusion criteria, or endpoint analysis for clinical trial design.

In various aspects, the present invention relates to a method of treating one or more of a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis with any of the agents described herein, including, without limitation, a compound of Formulae I-IV. In some embodiments, there is provided a method of treating diabetic retinopathy by administering a compound of Formulae I-IV to a patient in need thereof.

In various aspects, the present invention relates to a method of treating dry AMD or RPD, comprising administering to a patient in need thereof an effective amount of a compound of Formulae I-IV, and a complement factor D inhibitor, such as Lampilizumab.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### DETAILED DESCRIPTION OF THE INVENTION

The present invention relates to, in part, the surprising finding that a compound of Formulae I-IV (as described herein) has immunomodulatory effects, specifically on M1/M2 macrophage polarization, that makes it an effective agent for ocular disorders, such as dry AMD and RPD including, for example, protecting the retinal pigment epithelium (RPE).

Accordingly, in one aspect, the invention provides a method for treating or preventing an ocular disorder, comprising administering to a subject in need thereof an effective amount of an agent effective for the treatment of an ocular disorder in which such agent is an immunomodulatory agent. In some embodiments, the immunomodulatory agent targets macrophages. In some embodiments, the immunomodulatory agent modulates M1/M2 polarization. In some embodiments, the immunomodulatory agent modulates (e.g. reduces) M1 macrophage activity and/or M1 gene or protein expression in the subject and/or modulates (e.g. increases or maintains) M2 macrophage activity and/or M2 gene or protein expression in the subject. In some aspects, the invention relates to a method for treating or preventing an ocular disorder, comprising administering to a subject in need thereof an effective amount of an agent effective for the treatment of an ocular disorder, wherein the subject has abnormal (e.g. increased or decreased) expression or activity of one or more of CD64, IDO, SOCS1, CXCL10 Marco, Nos2, II12b, Ptgs2 (Cox2), II23a (II23p19), Ido1, Adipoq, Ccl20, IL17 (subtype a) Ccl5, CD163, Cx3Cr1, FasIg, Gfap, Csf2, Icam1, Ifng, II10, II12b, II13, II17, II18, II1b, II22, II4, II6, Klf4, Mrc1, Myd88, Nlrp3, Ppary, Tgfb1, Tlr4, Tnf, Vcam1, Ccl2, Ccl5, Ccl7, Ccr2, Socs1, Socs3, Stat1, Stat3, and Stat6.

In some embodiments, the subject has a modulated (e.g. decreased or increased) expression or activity of one or more of MRC1, TGM2, CD23, CCL22 Relma (Fizz1, Retnla), Socs2, Irf4, Chia (Amcase), Chi3I1 (Gp39, Ykl40), Chi3I2 (Yk139), Chi3I3 (Ym1), Cxcl13, Ccl12, Ccl24, and Klf4.

In some embodiments, the gene responses mitigated by an agent of the present invention (and therefore useful markers of the invention) are one or more of: Adipoq, ApoE, Timp3, Gfap, Nlrp3, Ccl11 (Eotaxin1), Cx3Cr1, Socs3, Stat1, Ccl20, II17a, RPE65, Cfb, C1qb, Serping1, C3, C4b, IL-6, Myd88, and C6.

In one aspect, the present invention provides a method for treating or preventing an ocular disorder, comprising administering to a subject in need thereof an effective amount of a MCP-modulating agent. In some embodiments, the MCP-modulating agent differentially targets the distal and/or proximal regulatory region of the MCP gene promoter/enhancer region(s). In some embodiments, the present invention provides a method for treating or preventing an ocular disorder, comprising administering to a subject in need thereof an effective amount of an MCP-1 and/or MCP-2 and/or MCP-3-modulating agent.

In some embodiments, the agent effective for the treatment of an ocular disorder is identified by the method of: (i) administering an effective amount of a test compound to an animal whose eye comprises (1) a fluorescent compound in an amount effective to indicate the presence of an ocular disorder in the animal and (s) a toxin in an amount effective to induce atrophy of ocular tissue; (ii) exposing the eye to light having a wavelength and intensity effective to cause the fluorescent compound to fluoresce; (iii) comparing the eye's fluorescence pattern to a fluorescence pattern of an animal's eye that comprises the fluorescent compound and the toxin, but not the test compound; and (iv) selecting the test compound as a candidate compound if the result of the comparison of step (iii) indicates that the test compound is useful for the treatment of an ocular disorder. In some embodiments, the agent effective for the treatment of an ocular disorder is an immunomodulatory agent, which optionally targets macrophages. In some embodiments, the agent effective for the treatment of an ocular disorder modulates (e.g. reduces) M1 macrophage activity and/or M1 gene or protein expression in the subject and/or modulates (e.g. increases) M2 macrophage activity and/or M2 gene or protein expression in the subject. In some embodiments, the agent effective for the treatment of an ocular disorder is an MCP-modulating agent, which optionally targets the gene promoter/enhancer region(s) of the MCP gene (optionally inclusive of the proximal promoter region of MCP). In some embodiments, the agent effective for the treatment of an ocular disorder is a small molecule with an indazole core. In some embodiments, the agent effective for the treatment of an ocular disorder is a compound of Formulae I-IV as shown herein.

In various aspects, the agent effective for the treatment of an ocular disorder of the present invention is one or more compounds described in one or more of the following documents the contents of which are hereby incorporated by reference in their entireties: International Patent Publication No. WO/2009/083436 (and U.S. Pat. Nos. 8,399,477 and 8,871,948); International Patent Publication No. WO/2009/109616 (and U.S. Pat. No. 8,461,194); International Patent Publication No. WO/2009/109618 (and U.S. Pat. Nos. 7,919,518 and 8,283,348); International Patent Publication No. WO/2009/109654 (and U.S. Pat. Nos. 8,314,099 and 8,569,297); International Patent Publication No. WO/2009/109613 (and U.S. Pat. No. 8,835,481); and International Patent Publication No. WO/2011/015501 (and U.S. Pat. No. 8,354,544).

In some embodiments, the ocular disorder is one or more of dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), retinal degeneration, late onset retinal degeneration (LORDs; i.e. Q1qTNF5 deficiency), and central serous retinopathy (CSR). In some embodiments, the ocular disorder is Lecithin Retinol Acyltransferase (LRAT) deficiency, which is optionally associated with: Irat-related leber congenital amaurosis, and retinal dystrophy, early-onset, severe. In some embodiments, the ocular disorder is fundus albipunctatus, which may be associated with one or more of the following genetic locations: 3q22.1 (Retinitis punctata albescens, RHO); 6p21.1 (Retinitis punctata albescens, PRPH2); 12q13.2 (Fundus albipunctatus, RDH5); 15q26.1 (Retinitis punctata albescens, RLBP1); and 15q26.1 (Fundus albipunctatus, RLBP1).

In some embodiments, the ocular disorder is one or more of a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; idiopathic uveitis, Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis.

In some embodiments, the subject is not undergoing treatment with and/or would benefit from therapy with and/or is unresponsive to one or more of an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). In some embodiments, the subject has evidence of AMD as confirmed by the presence of at least 1 druse greater than about 125 μm in diameter. In some embodiments, the subject may or may not have evidence of prior or concurrent active choroidal neovascularization (CNV). In some embodiments, the subject has one or more well-demarcated GA lesions of a total area of about 2 to about 20 mm² in one or more eye and/or 0.5 to 10 disc areas (e.g. 0.5 to 7, or about 0.5, or about 1, or about 2, or about 3, or about 4, or about 5, or about 6, or about 7, or about 8, or about 9, or about 10 disc areas). In some embodiments, the subject has a best-corrected visual acuity score of greater than about 35 letters or a Snellen VA equivalent of about 20/200 or better. In some embodiments, the subject has early stage dry macular degeneration as evidenced by a large number of small drusen or a few medium-sized drusen. In some embodiments, the subject has early stage dry macular degeneration as evidenced by a large number of medium-sized drusen or one or more large drusen. In some embodiments, the subject has early stage dry macular degeneration as evidenced by several large drusen and/or an extensive breakdown of cells in the macula. In some embodiments, the subject has early stage dry macular degeneration as evidenced by large drusen and pigmentary change.

In some embodiments, the method further comprises administering an additional therapeutic agent. The additional therapeutic agent may be, in various embodiments, an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). The additional therapeutic agent may also be, in various embodiments, an anti-vascular endothelial growth factor (VEGF) agent, an angiotensin-converting enzyme (ACE) inhibitor, a peroxisome proliferator-activated receptor (PPAR)-gamma agonist, a renin inhibitor, a steroid, and an agent that modulates autophagy. In some embodiments, the additional therapeutic agent is a modulator of the complement cascade (e.g. a modulator of C3, C5, complement factor D, or complement factor B).

In some embodiments, the additional therapeutic agent is a nucleoside reverse transcriptase inhibitor (NRTIs), by way of non-limiting example zidovudine, didanosine, zalcitabine, stavudine, lamivudine, abacavir, emtricitabine, and entecavir. In some embodiments, the additional therapeutic agent is acyclovir.

In one aspect, the present invention provides a method of making an agent effective for the treatment of an ocular disorder, comprising (a) identifying the agent by (i) administering an effective amount of a test compound to an animal whose eye comprises (1) a fluorescent compound in an amount effective to indicate the presence of an ocular disorder in the animal and (2) a toxin in an amount effective to induce atrophy of ocular tissue; (ii) exposing the eye to light having a wavelength and intensity effective to cause the fluorescent compound to fluoresce; (iii) comparing the eye's fluorescence pattern to a fluorescence pattern of an animal's eye that comprises the fluorescent compound and the toxin, but not the test compound; and (iv) selecting the test compound as a candidate compound if the result of the comparison of step (iii) indicates that the test compound is useful for the treatment of an ocular disorder and (b) formulating the identified agent for administration to the eye. In some aspects, there is provided a method for treating or preventing an ocular disorder, comprising administering to a subject in need thereof a pharmaceutical composition comprising an effective amount of an agent or any one of the agent effective for the treatment of an ocular disorder as made in using the method described above and a pharmaceutically acceptable carrier or excipient.

In some embodiments, the agent effective for the treatment of an ocular disorder is an immunomodulatory agent, which optionally targets macrophages. In some embodiments, the agent effective for the treatment of an ocular disorder modulates (e.g. reduces) M1 macrophage activity and/or M1 gene and protein expression in the subject and/or modulates (e.g. increases) M2 macrophage activity and/or M2 gene and protein expression in the subject.

In some embodiments, the agent effective for the treatment of an ocular disorder is an MCP-modulating agent, which optionally targets the distal and/or proximal regulatory region of the MCP gene promoter/enhancer region(s).

In some embodiments, the agent effective for the treatment of an ocular disorder is a compound of

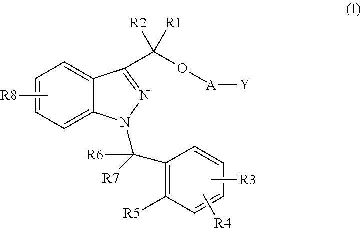

or a pharmaceutically acceptable salt thereof, wherein:

A may be a bond a, —X₁— or —X₁—O—X₂—, in which

X₁ and X₂, which may be identical or different from each other, may be an alkyl group having from 1 to 5 carbon atoms, optionally substituted with one or more alkyl groups having from 1 to 5 carbon atoms or one or more alkoxy groups having from 1 to 3 carbon atoms,

Y is H when A is a bond a, or Y may be H, —OH, or —N(R₁₁)(R₁₂), when A is —X₁— or —X₁—O—X₂—, in which

R₁₁ may be hydrogen, an alkyl group having from 1 to 5 carbon atoms, or R₁₁ together with R₁₂ forms a 4- to 7-membered heterocycle,

R₁₂ may be hydrogen, an alkyl group having from 1 to 5 carbon atoms, or R₁₂ together with R₁ forms a 4- to 7-membered heterocycle,

R₁ and R₂, which may be identical or different from each other, may be hydrogen, an alkyl group having from 1 to 5 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms, and

R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms.

In certain embodiments, the agent effective for the treatment of an ocular disorder is a compound of Formula I with the proviso that when A is a a bond, and Y, R₁, R₂, R₆, and R₇ are hydrogen atoms,

if R₈ is a hydrogen atom, then the group linked to the nitrogen atom in the 1-position of the indazole ring is different from a benzyl group, a 4-chlorobenzyl group, or a 2-4-dichlorobenzyl group,

if R₈ is a fluorine atom in the 5-position of the indazole ring, then the group linked to the nitrogen atom in the 1-position of the indazole ring is different from 5-chloro-2-methoxybenzyl group, and

if R₈ is a trifluoromethyl group in the 6-position of the indazole ring, then the group linked to the nitrogen atom in the 1-position of the indazole ring is different from a 2-4-dichlorobenzyl group.

In some embodiments, the compound of Formula I is selected from:
1-benzyl-3-hydroxymethyl-indazole;
1-(4-methoxybenzyl)-1H-indazol-3-yl]methanol;
1-(4-methylbenzyl)-1H-indazol-3-yl]methanol;
1-(4-chlorobenzyl)-1H-indazol-3-yl]methanol;
1-(3,4-dichlorobenzyl)-1H-indazol-3-yl]methanol;
1-(2,4-dichlorobenzyl)-1H-indazol-3-yl]methanol;
1-(4-fluorobenzyl)-1H-indazol-3-yl]methanol;
1-(4-chloro-2-methylbenzyl)-1H-indazol-3-yl]methanol;
1-benzyl-5-methoxy-1H-indazol-3-yl)methanol;
1-benzyl-5-methoxy-1H-indazole-3-carboxylate;
2-[1-(4-chloro-2-methylbenzyl)-1H-indazol-3-yl]propan-2-ol;
1-(4-chloro-2-methylbenzyl)-1H-indazole-3-carboxylate;
2-[1-(2,4-dichlorobenzyl)-1H-indazol-3-yl]propan-2-ol;
1-(2,4-dichlorobenzyl)-1H-indazole-3-carboxylate;
1-[1-(4-chloro-2-methylbenzyl)-1H-indazol-3-yl]ethanol;
1-(4-chloro-2-methylbenzyl)-1H-indazole-3-carboxaldehyde;
1-[1-(4-chloro-2-methylbenzyl)-1H-indazol-3-yl]ethanol
1-(4-chloro-2-methylbenzyl)-1H-indazole-3-carboxaldehyde;
2-[(1-benzyl-1H-indazol-3-yl)methoxy]ethanol;
2-[(1-benzyl-1H-indazol-3-yl)methoxy]-N,N-dimethylethanamine hydrochloride;
3-[(1-benzyl-1H-indazol-3-yl)methoxy]-N,N-dimethylpropan-1-amine hydrochloride;
3-[(1-benzyl-1H-indazol-3-yl)methoxy]propan-1-ol;
2-[(1-benzyl-1H-indazol-3-yl)methoxy]-2-methylpropan-1-ol;
1-benzyl-3-[(1,1-dimethyl-2-morpholin-4-ylethoxy)methyl]-1H-indazole maleate; and
1-benzyl-3-[(1,1-dimethyl-2-morpholin-4-yl-2-oxyethoxy)methyl]-1H-indazole.

In some embodiments, the agent effective for the treatment of an ocular disorder is a compound of Formula II:

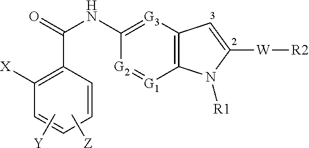

or a pharmaceutically acceptable salt thereof, wherein:

X is a halogen atom or a (C₁-C₃)alkyl, trifluoromethyl, nitro, amino, cyano, di(C₁-C₃)alkylamino, hydroxy, (C₁-C₃)alkoxy, phenyl or (C₁-C₃)alkylphenyl group; Y and Z, which may be identical or different, are a hydrogen or halogen atom, or a (C₁-C₃)alkyl, trifluoromethyl, nitro, amino, di(C₁-C₃)alkylamino, hydroxy, (C₁-C₃)alkoxy, phenyl, COOH, (C₁-C₃)alkyl-COOH, (C₂-C₃)alkenyl-COOH, COOR, wherein R is a linear or branched (C₁-C₆)alkyl or hydroxyalkyl group, CONH₂, SO₂CH₃, SO₂NHCH₃ or NHSO₂CH₃ group;

G1, G2, and G3, which may be identical or different, are a nitrogen atom or a CH group;

R1 is a (C₁-C₆)alkyl, (C₃-C₇)cycloalkyl, (C₁-C₆)alkylOR^{I}, (CH₂)ₙNR^{II}R^{III}, (CH₂)ₙCONR^{II}R^{III}, (CH₂)ₙCOR^{I}, (CH₂)ₙCOOR^{II}, (CH₂)ₙOCOR^{I}, SO₂R^{I}, (CH₂)ₙNR^{II}SO₂R^{I}, (CH₂)ₙSO₂R^{I} group, optionally substituted with 1 to 3 hydroxy groups, wherein n is an integer from 1 to 6, R^{I} is a (C₁-C₃)alkyl, or (C₁-C₃)alkylOH group, and R^{II} and R^{III}, which may be identical or different, are a hydrogen atom or a (C₁-C₃)alkyl group;

W is a σ bond, or a (C₁-C₆)alkyl, (C₂-C₆)alkenyl, O(C₁-C₆)alkyl, O(C₂-C₆)alkenyl, C(O)NH, (CH₂)ₚCO(CH₂)_{q}, or (CH₂)ₚC(OH)(CH₂)_{q} group,

wherein p and q, which may be identical or different, are an integer from 0 to 3;

R2 is a phenyl, pyridine or (C₃-C₇)cycloalkyl group, optionally substituted with 1 to 3 substituents, which may be identical or different, represented by a L-M group, wherein L is a σ bond, or a (C₁-C₆)alkyl, (C₂-C₆)alkenyl, (C₂-C₆)alkinyl, O(C₁-C₆)alkyl, O(C₂-C₆)alkenyl, O(C₂-C₆)alkinyl group, and M is a hydrogen or halogen atom, or a OH, CF₃, NO₂, ON, COOR^{II}, SO₂NHR^{II}, CH₂CONR^{II}R^{III}, NR^{II}R^{III}, SO₂R^{IV}, NHSO₂R^{IV}, POR^{IV}R^{V}, or OPOR^{IV}R^{V} group, wherein R^{II} and R^{III}, which may be identical or different, have the meaning above, and R^{IV} and R^{V}, which may be identical or different, are a (C₁-C₃)alkyl group,

provided that

when G1, G2, and G3 are all a CH group, R1 is a (C₁-C₆)alkyl or (C₃-C₇)cycloalkyl group, optionally substituted with 1 to 3 hydroxy groups, W is a σ bond, and the bond between the carbon atoms in the 2 and 3 position is a double bond,

R2 is not a phenyl or pyridine group, optionally substituted with 1 to 3 substituents, which may be identical or different, selected from halogen, (C₁-C₆)alkyl optionally substituted with a hydroxy group, trifluoromethyl, nitro, amino, di(C₁-C₃)alkylamino, hydroxy, (C₁-C₃)alkoxy, COOH, COOR^{II}, SO₂CH₃, SO₂NHCH₃, NHSO₂CH₃, POR^{IV}R^{V}, OPOR^{IV}R^{V}, (C₁-C₆)alkyl-COOH, and (C₂-C₆)alkenyl-COOH,

and provided that when G1 is N, and G2 and G3 are a CH group, R2 is not a divalent aromatic group substituted with one L-M group represented by O(C₁-C₆)alkyl, O(C₂-C₆)alkenyl, and O(C₂-C₆)alkinyl group.

In some embodiments, the compound of Formula II is selected from:
2-[(1-benzyl-1H-indazol-3-yl)methoxy]-2-methylpropanamide;
methyl 2-[(1-benzyl-1H-indazol-3-yl)methoxy]-2-methylpropanoate;
2-[(1-benzyl-1H-indazol-3-yl)methoxy]-N-hydroxy-2-methylpropanamide;
methyl 2-[(1-benzyl-1H-indazol-3-yl)methoxy]-2-methylpropanoate;
2-[(1-benzyl-1H-indazol-3-yl)methoxy]-2-methylpropanohydrazide;
2-{2-[(1-benzyl-1H-indazol-3-yl)methoxy]-2-methylpropanoyl}hydrazine-carboxamide;
2-[(1-benzyl-1H-indazol-3-yl)methoxy]-2-methyl-N-pyrid-2-ylpropanamide;
1-benzyl-3-{[1,1-dimethyl-2-(4-methylpiperazin-1-yl)-2-oxoethoxy]methyl)-1H-indazole;
N-{2-[(1-benzyl-1H-indazol-3-yl)methoxy]-2-methylpropanoyl}glycine;
2-[(1-benzyl-1H-indazol-3-yl)methoxy]-2-methyl-N′-phenylpropanohydrazide;
1-benzyl-3-[(1,1-dimethyl-2-morpholin-4-il-2-oxoethoxy)methyl]-1H-indazole;
2-[(1-benzyl-5-methoxy-1H-indazol-3-yl)methoxy]-2-methylpropanamide;
2-{[1-(4-chlorobenzyl)-1H-indazol-3-yl]methoxy}-2-methylpropanamide;
[1-(4-chlorobenzyl)-1H-indazol-3-yl]methanol;
2-{[1-(3,4-dichlorobenzyl)-1H-indazol-3-yl]methoxy}-2-methylpropanamide;
[1-(3,4-dichlorobenzyl)-1H-indazol-3-yl]methanol;
2-[(1-benzoyl-1H-indazol-3-yl)methoxy]-N-hydroxy-2-methylpropanamide;
(1-tritylindazol-3-yl)methanol;
2-methyl-2-{[1-(1,2,3,4-tetrahydronaphth-1-yl)-1H-indazol-3-yl]methoxy)-propanoic acid;
2-{[1-(1,2,3,4-tetrahydronaphth-1-yl)-1H-indazol-3-yl]methoxy}-N-hydroxy-2-methylpropanamide;
{[1-(4-methoxybenzyl)-1H-indazol-3-yl]methoxy)-N-hydroxyacetamide 36a) [1-(4-methoxybenzyl)-1H-indazol-3-yl]methanol;
{2-[(1-benzyl-1H-indazol-3-yl)methoxy]-2-methylpropoxy}acetamide 39a) 2-[(1-benzyl-1H-indazol-3-yl)methoxy]-2-methylpropan-1-ol;
{2-[(1-benzyl-1H-indazol-3-yl)methoxy]-2-methylpropoxy)-N-hydroxy-acetamide;
(2-{[1-(4-methoxybenzyl)-1H-indazol-3-yl]methoxy}-2-methylpropoxy)-N-hydroxy-acetamide 41a) [1-(4-methoxybenzyl)-1H-indazol-3-yl]methanol;
2-{2-[(1-benzyl-1H-indazol-3-yl)methoxy]ethoxy}-N-hydroxy-2-methylpropanamide;
2-{3-[(1-benzyl-1H-indazol-3-yl)methoxy]propoxy}-N-hydroxy-2-methylpropanamide;
3-[(1-benzyl-1H-indazol-3-yl)methoxy]propan-1-ol;
{2-[(1-benzyl-1H-indazol-3-yl)methoxy]ethoxy}acetamide;
2-[(1-benzyl-1H-indazol-3-yl)methoxy]ethanol;
{2-[(1-benzyl-1H-indazol-3-yl)methoxy]ethoxy}-N-hydroxy-acetamide; and
{2-[(1-benzyl-1H-indazol-3-yl)methoxy]ethoxy}-acetohydrazide.

In some embodiments, the agent effective for the treatment of an ocular disorder is a compound of Formula III:

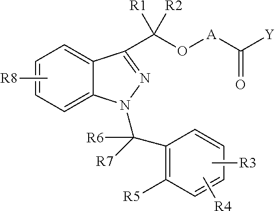

or a pharmaceutically acceptable salt thereof, wherein:

A may be —X₁ or —X₁—OC(R₉)(R₁₀)—, in which
X₁ may be an alkyl group having from 1 to 5 carbon atoms, optionally substituted with one or more alkyl groups having from 1 to 5 carbon atoms or one or more alkoxy groups having from 1 to 3 carbon atoms, and
R₉ and R₁₀, which may be identical or different from each other, may be hydrogen, an alkyl group having from 1 to 5 carbon atoms or an alkoxy group having from 1 to 3 carbon atoms,

Y may be N(R₁₁)(R₁₂), N(R₁₃)O(R₁₄), N(R₁₃)N(R₁₄)(R₁₅), N(R₁₃)—X₂—N(R₁₄)(R₁₅), N(R₁₃)—X₂—CO—X₃, in which
R₁₁ may be hydrogen, an alkyl group having from 1 to 5 carbon atoms, an alkoxy group having from 1 to 3 carbon atoms, or R₁₁ together with R₁₂ forms a 4- to 7-membered heterocycle,
R₁₂ may be hydrogen, an alkyl group having from 1 to 5 carbon atoms, an alkoxy group having from 1 to 3 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms, or R₁₂ together with R₁₁, forms a 4- to 7-membered heterocycle,
R₁₃ and R₁₅, which may be identical or different from each other, may be hydrogen, an alkyl group having from 1 to 5 carbon atoms or an alkoxy group having from 1 to 3 carbon atoms,
R₁₄ may be hydrogen, an alkyl group having from 1 to 5 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms,
X₂ may be an alkyl group having from 1 to 5 carbon atoms, optionally substituted with one or more alkyl groups having from 1 to 5 carbon atoms or one or more alkoxy groups having from 1 to 3 carbon atoms, X₃ may be OH, NH₂, NHOH or NHNH₂,

R₁ and R₂, which may be identical or different from each other, may be hydrogen, an alkyl group having from 1 to 5 carbon atoms or an alkoxy group having from 1 to 3 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R′)(R″), —N(R′)COR″, nitro and trifluoromethyl, or R₅ together with one from among R₆ and R₇ forms a ring having 5 or 6 carbon atoms; with R′ and R″, which may be identical or different from each other, represented by hydrogen and an alkyl group having from 1 to 5 carbon atoms,

R₆ and R₇, which may be identical or different from each other, may be hydrogen, an alkyl group having from 1 to 5 carbon atoms, or together form a group C═O, or one from among R₆ and R₇, together with R₅, forms a ring having 5 or 6 carbon atoms.

In some embodiments, the agent effective for the treatment of an ocular disorder is a compound of Formula IV:

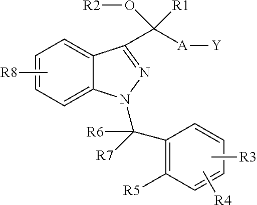

or a pharmaceutically acceptable salt thereof, wherein:

A may be —X₁— or —X—O—X₂—, in which
X₁ may be an alkyl group having from 1 to 5 carbon atoms, optionally substituted with one or more alkyl groups having from 1 to 5 carbon atoms or one or more alkoxy groups having from 1 to 3 carbon atoms, and
X₂ may be an alkyl group having from 1 to 5 carbon atoms, optionally substituted with one or more alkyl groups having from 1 to 5 carbon atoms,

Y may be hydrogen, —OH, —N(R₁₁)(R₁₂), —N(R₁₁)O(R₁₂), in which
R₁₁ may be hydrogen, an alkyl group having from 1 to 5 carbon atoms, an alkoxy group having from 1 to 3 carbon atoms, or R₁₁ together with R₁₂ forms a 4- to 7-membered heterocycle,
R₁₂ may be hydrogen, an alkyl group having from 1 to 5 carbon atoms, an alkoxy group having from 1 to 3 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms, or R₁₂ together with R₁₁, forms a 4- to 7-membered heterocycle,

R₁ and R₂, which may be identical or different from each other, may be hydrogen, an alkyl group having from 1 to 5 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R′)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms,

R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms.

In some embodiments, the compound of Formula IV is selected from:
2-amino-1-(1-benzyl-1H-indazol-3-yl)ethanol and
1-(1-benzyl-1H-indazol-3-yl)-2-(ethylamino)ethanol.

In various aspects, the present methods are applicable to select subject populations. For example, the present methods, including embodiments in which the agent is a compound of Formulae I-IV, and the ocular disorder is dry AMD or RPD, the subject is not undergoing treatment with and/or is unresponsive to one or more of an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). In various embodiments, including embodiments in which the agent is a compound of Formulae I-IV and the ocular disorder is dry AMD or RPD, the subject is classified by having one or more of the following: at least 1 druse greater than about 125 μm in diameter, no evidence of prior or active choroidal neovascularization (CNV), one or more well-demarcated GA lesions of a total area of about 2 to about 20 mm² in one or more eye, a best-corrected visual acuity score of greater than about 35 letters or a Snellen VA equivalent of about 20/200 or better, and a macular photocoagulation study disc area of less than about 7.0, and less than 1 disc up to more than 10 disc areas (e.g. about 1, or about 2, or about 3, or about 4, or about 5, or about 6, or about 7, or about 8, or about 8, or about 9, or about 10 disc areas). In various embodiments, the subject is classified as having one or more of: at least 1 large druse, or RPD, and hypofluorescent (dark or black) regions identified by DNIRA of a total area of about 2 mm² to about 20 mm² in one or more eye, or less than 1 disc areas or up to more than 10 disc areas (e.g. about 1, or about 2, or about 3, or about 4, or about 5, or about 6, or about 7, or about 8, or about 8, or about 9, or about 10 disc areas) of hypofluorescent DNIRA signal, in one or more eye.

In one aspect, the invention provides a method for treating or preventing an ocular disorder, comprising administering to a subject in need thereof an effective amount of a compound of Formulae I-IV as described herein. In some embodiments, the ocular disorder is one or more of dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR). In some embodiments, the ocular disorder is one or more of a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis. In some embodiments, the invention provides a method for treating or preventing an ocular disorder, comprising administering to a subject in need thereof an effective amount of a compound of Formulae I-IV or a pharmaceutically acceptable salt thereof, wherein the ocular disorder is selected from a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis.

In a further aspect, the invention provides a method for treating or preventing an ocular disorder, comprising administering to a subject in need thereof an effective amount of a compound of Formulae I-IV or a pharmaceutically acceptable salt thereof, wherein the ocular disorder is selected from a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis.

In some embodiments, the patient has type 1 or type 2 diabetes. In various embodiments, the patient has a blood glucose level that exceeds normal (about 70-130 milligrams per deciliter or mg/dL before meals, and less than 180 mg/dL one to two hours after a meal).

In other embodiments, the present invention pertains to treatment or prevention of a cataract or glaucoma, comprising administering to a patient in need thereof an effective amount of a compound of Formulae I-IV, or a pharmaceutically acceptable salt thereof.

In some embodiments, the methods further comprise administering an additional therapeutic agent. In various embodiments, the additional therapeutic agent is one or more of an anti-vascular endothelial growth factor (VEGF).

In one aspect, the invention provides methods for identifying whether an agent is effective for the treatment of an ocular disorder, including a diabetic eye disease (by way non-limiting example, diabetic retinopathy and DME), comprising (a) administering an effective amount of a test compound to an animal whose eye comprises (i) a fluorescent compound in an amount effective to indicate the presence of ocular disorder, including a diabetic eye disease (by way non-limiting example, diabetic retinopathy and DME) disease in the animal and (ii) a toxin in an amount effective to induce atrophy of ocular tissue; (b) exposing the eye to light having a wavelength and intensity effective to cause the fluorescent compound to fluoresce; (c) comparing the eye's fluorescence pattern to a fluorescence pattern of an animal's eye that comprises the fluorescent compound and the toxin but not the test compound; and (d) selecting the test compound as a candidate compound if the result of the comparison of step (c) indicates that the test compound is useful for the treatment of an ocular disorder. In some embodiments, the method provides a further step of making an agent effective for treating an ocular disorder by formulating the candidate compound for ocular delivery.

In some embodiments, the ocular disorder is one or more of a diabetic eye disease (by way non-limiting example, diabetic retinopathy and DME), AMD, RPD disease Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis.

In some embodiments, the animal is any one of those listed herein, such as, by way on non-limiting example, a mouse, rat, zebrafish, dog, cat, pig, horse or non-human primate.

In other embodiments, the fluorescent compound absorbs light at a wavelength of about 600 nm to about 900 nm and/or emits light at a wavelength of about 750 nm to about 950 nm. In a specific embodiment, the fluorescent compound is ICG. In still other embodiments, the fluorescence occurs in RPE cells.

In some embodiments, the toxin is sodium iodate. In other embodiments, the atrophy comprises necrosis of RPE cells. In other embodiments, the necrosis presents as patches.

In still other embodiments, the comparing occurs at least about 24 hours, or at least about 7 days, or at least about 30 days, or at least 60 days, or at least 90 days after administering the test compound.

In various embodiments, the exposing the eye to light comprises performing cSLO, FAF, DNIRA, IR, or OCT. In various embodiments, the exposing the eye to light comprises white light, blue light, red-free light, near infra-red, or infra-red.

In various embodiments, the presence of an ocular disorder is indicated by patterns of FAF within patches of RPE damage or loss or outer retinal loss. In various embodiments, the presence of an ocular disorder is indicated by patterns of FAF that occur within, or adjacent to, or in proximity to, or distant from, or in the absence of patches of RPE damage or loss or outer retinal loss. In some embodiments, the patterns are one or more of curvilinear, ribbon-like, reticular, oval, circular, scalloped, halo, and target-like lesions. In various embodiments, the presence of an ocular disorder is indicated by patterns of FAF at the border of patches of RPE damage or loss or outer retinal loss. In some embodiments, the patterns are one or more of curvilinear, ribbon-like, reticular, oval, circular, scalloped, halo, and target-like lesions. In various embodiments, the presence of an ocular disorder is indicated by patterns of FAF that occur distant from patches of RPE damage or loss (in the so-called junctional zone), or in the absence of patches of RPE damage or loss or outer retinal loss. In some embodiments, the patterns are one or more of curvilinear, ribbon-like, reticular, oval, circular, scalloped, halo, and target-like lesions.

In various other embodiments, the presence of an ocular disorder is indicated by cross-sectional patterns or transverse patterns. In some embodiments, the patterns are observed with OCT. In some embodiments, the patterns are RPE and/or outer retinal loss or mounds, triangles, peaks or spikes found in the sub-retinal space. In some embodiments, for example in the case of LORDs, illustrative symptoms/diagnostic parameters include scalloped areas of retinal pigment epithelium (RPE) atrophy in the mid-periphery and widespread atrophy in the posterior pole (e.g. as observed by dilated fundus examination); rod-cone dystrophy (e.g. as observed by ERG); widespread loss of the photoreceptors with absence of the inner/outer segment junction line and concurrent thinning of the outer nuclear layer (e.g. as seen by SD-OCT), diffuse choroidal thinning, including affecting the inner choroid with loss of the choriocapillaris.

In still other embodiments, the methods further comprise the step of observing the eye prior to administering the test compound. In some embodiments, this observing establishes one or more pre-administration characteristics of the eye.

In yet another embodiment, the methods described herein comprise administering the fluorescent compound prior to administering the test compound. In still another embodiment, the methods described herein do not comprise administering (i) an additional amount of a fluorescent compound to the animal or (ii) a second fluorescent compound to the animal. In other embodiments, the methods described herein comprise administering the toxin prior to administering the test compound and/or administering the toxin prior to administering the fluorescent compound.

In still another embodiment, the methods described herein comprise administering (i) an additional amount of the toxin to the animal or (ii) a second toxin to the animal. In still another embodiment, the methods described herein comprise administering (i) an additional amount of the toxin to the animal, (ii) a second toxin to the animal, or (iii) a compound believed to influence the mechanism of action of the toxin. In some embodiments, the methods described herein comprise further comprise observing a reduction in the rate of formation, growth or expansion of patches of ocular tissue atrophy or patches of tissue loss.

In some embodiments, the candidate compound is useful for treating, preventing, or reducing the rate of pathogenesis of an ocular disorder. In other embodiments, a plurality of candidate compounds is identified. In some embodiments, the methods described herein further comprise comparing the usefulness of the plurality of candidate compounds in the treatment of an ocular disorder and selecting a lead compound based on the comparison.

In some embodiments, the present invention pertains to prevention or treatment of dry AMD, which may be is identifiable by the presence of areas of hyper-fluorescent FAF in an eye of the subject and/or the presence of one or more areas of abnormally fluorescent FAF in an eye of the subject and/or by changes in one or more of blue spectrum fundus imaging, white-light fundus imaging, red-free fundus imaging, and OCT in an eye of the subject and/or by an increase (including a transient increase) in permeability across the subject's epithelial barrier between a choroid and a retina relative to an undiseased state and/or by a deformation of the outer retina, and/or deformation of the mid-retinal vasculature across the subject's epithelial barrier between a choroid and a retina relative to an undiseased state and/or by the presence of phagocytic immune cells across the subject's RPE relative to an undiseased state.

In some embodiments, the dry AMD is early stage AMD, or is late stage atrophic dry AMD.

In other embodiments, the subject is a human. In still other embodiments, the administering is effected orally or intra-vascularly, or intraocularly, or periocularly, or to the ocular surface.

In another aspect, the invention provides a method of treating RPD disease, comprising administering to a subject in need thereof an effective amount of a compound of Formulae I-IV (as described herein) or a pharmaceutically acceptable salt thereof.

In some embodiments, the methods described herein comprise reducing the amount of pseudodrusen in the subject and/or reducing the amount of pseudodrusen in any one of the foveal area, perifoveal area, juxtafoveal area, and extrafoveal area of the subject's eye. In other embodiments, the methods described herein comprise reducing the rates of progression to late disease, wherein the late disease is any one of choroidal neovascularization or geographic atrophy. In some embodiments, the methods described herein comprise reducing the rates of expansion of geographic atrophy.

In some embodiments, the methods described herein comprise administering an additional therapeutic agent. In various embodiments, the additional therapeutic is one or more of an anti-vascular endothelial growth factor (VEGF) agent, a modulator of the complement cascade, an angiotensin-converting enzyme (ACE) inhibitor, a peroxisome proliferator-activated receptor (PPAR)-gamma agonist, a renin inhibitor, a corticosteroid, and an agent that modulates autophagy.

In still other embodiments, the RPD disease is identifiable by the presence of one or more areas of distinct patterns of retinal imaging in the eye of a subject, wherein the retinal imaging is one or more of white light, red-free light, blue light, FAF, near infra-red (NIR), infra-red (IR), angiography, and DNIRA and/or the presence of one or more areas of abnormally-fluorescent FAF in the eye of a subject and/or an increase (including a transient increase) in permeability across the subject's epithelial barrier between a choroid and a retina relative to an undiseased state and/or a presence of phagocytic immune cells across the subject's RPE relative to an undiseased state.

In other embodiments, the subject is a human. In still other embodiments, the administering is effected orally or intra-vascularly, or intraocularly, or periocularly, or to the ocular surface.

In other embodiments, the invention provides for the use of compounds of Formulae I-IV, or their pharmaceutically acceptable salts, alone or in combination with an additional therapeutic, in the manufacture of a medicament useful for the treatment or prevention of one or more ocular disorders.

In yet another aspect, the invention provides a method for identifying a subject who has an ocular disorder and is more likely than not to respond to treatment with an agent comprising determining whether the subject's eye has, or previously had, an increase (including a transient increase) in permeability across the epithelial barrier between a choroid and a retina of the eye relative to an undiseased state; wherein the increase in permeability indicates that the subject is more likely than not to respond to treatment with the agent; and wherein the agent is selected from a compound of Formulae I-IV (as described herein) or a pharmaceutically acceptable salt thereof.

In some embodiments, the ocular disorder is one or more of a diabetic eye disease, dry AMD and RPD disease.

In other embodiments, the diabetic eye disease is diabetic retinopathy or DME.

In still another aspect, the present invention provides a method for identifying an ocular disorder subject who is more likely than not to respond to treatment with an agent comprising determining whether the subject's eye has a presence of phagocytic immune cells (optionally derived from within the retina, the RPE, the choroid, or the blood stream) across the RPE relative to an undiseased state, wherein the presence of phagocytic immune cells indicates that the subject is more likely than not to respond to treatment with the agent; and wherein the agent is selected from a compound of Formulae I-IV (as described herein) or a pharmaceutically acceptable salt thereof.

In some embodiments, the ocular disorder is one or more of dry AMD and RPD disease. In other embodiments, the presence of phagocytic immune cells is measured by DNIRA.

In another aspect, the invention provides a method for determining whether an ocular disorder in a subject is responsive to treatment with an agent that inhibits or modifies the function of a subject's immune cells, comprising detecting a presence, detecting an absence, or measuring an amount of immune cells in the subject's eye, wherein the subject's eye fluoresces in response to light having a wavelength of about 600 nm to about 900 nm. In some embodiments, the light has a wavelength of about 400 nm to about 900 nm

In some embodiments, the methods described herein further comprise administering to the subject an effective amount of a fluorescent compound, wherein the detecting or measuring occurs at least one day after the administration of the fluorescent compound. In some embodiments, the detecting or measuring occurs at least one day after administering to the subject an effective amount of a fluorescent compound.

In some embodiments, the methods described herein further comprise the step of detecting or measuring FAF in the eye of the subject. In some embodiments, the methods described herein further comprise the step of correlating an FAF pattern to the presence, absence, or amount of immune cells in the subject's eye.

In other embodiments, the ocular disorder is a diabetic eye disease, AMD, central serous retinopathy (CSR) or RPD disease. In some embodiments, the subject is a human.

In other embodiments, the diabetic eye disease is diabetic retinopathy or DME.

In various embodiments, the subject's eye fluoresces light having a wavelength of about 750 nm to about 950 nm. In some embodiments, the fluorescent compound is ICG.

In some embodiments, the detecting or measuring occurs at about one day, or about seven days, or at about thirty days after administration of the fluorescent compound.

In some embodiments, the methods described herein do not further comprise administering (a) an additional amount of the fluorescent compound or (b) a second fluorescent compound.

In other embodiments, the detecting or measuring comprises performing cSLO, FAF, DNIRA or OCT.

In some embodiments, the immune cells are cells of the subject's innate immune system and/or macrophage and/or microglial cells.

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with a test compound and/or candidate compound is an amount that is effective for providing a measurable treatment, prevention, or reduction in the rate of pathogenesis of an ocular disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis, AMD and/or RPD.

An “effective amount,” when used in connection with a fluorescent compound is an amount that allows optical detection.

An “effective amount,” when used in connection with an agent effective for the treatment of an ocular disorder, a compound of Formulae I-IV, or a pharmaceutically acceptable salt thereof, is an amount that is effective for treating or preventing an ocular disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis, AMD and/or RPD.

An “effective amount,” when used in connection with another therapeutic agent is an amount that is effective for treating or preventing an ocular disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis, AMD and/or RPD (e.g. providing a measurable treatment, prevention, or reduction in the rate of pathogenesis) alone or in combination with a compound of Formulae I-IV, or a pharmaceutically acceptable salt thereof. “In combination with” includes administration within the same composition and via separate compositions; in the latter instance, the other therapeutic agent is effective for treating or preventing a condition during a time when the agent a compound of Formulae I-IV, or a pharmaceutically acceptable salt thereof, exerts its prophylactic or therapeutic effect, or vice versa.

An “effective amount,” when used in connection with a toxin, for example, sodium iodate, is an amount that is effective for inducing measurable atrophy of ocular tissue as described herein.

An agent is “useful for the treatment of an ocular disorder” if the agent provides a measurable treatment, prevention, or reduction in the rate of pathogenesis of an ocular disorder.

The term “ocular disorder” refers to one of various ophthalmic diseases and includes diseases of the eye and the ocular adnexa. The term “ocular disorder” includes, for example, disorders described herein (for instance, by way of non-limiting example, an ocular disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis, wet AMD, dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR).

The term “neovascularization” refers to new blood vessel formation in abnormal tissue or in abnormal positions.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-A/VPF gene including VEGF₁₂₁, VEGF₁₆₅ and VEGF₁₈₉. Further, as used herein, the term “VEGF” includes VEGF-related angiogenic factors such as PIGF (placental growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Fit-1), VEGFR-2 (KDR/Flk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF₁₆₅. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

The term “steroid” refers to compounds belonging to or related to the following illustrative families of compounds: corticosteroids, mineralocosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others.

The terms “peroxisome proliferator-activated receptor gamma agent,” or “PPAR-γ agent,” or “PPARG agent,” or “PPAR-gamma agent” refers to agents which directly or indirectly act upon the peroxisome proliferator-activated receptor. This agent may also influence PPAR-alpha, “PPARA” activity.

The term “an agent that modulates autophagy” refers to a modulator of cell survival, cell death, survival, autophagy, proliferation, regeneration, and the like.

The term “monocyte chemotactic protein,” or “MCP” refers to the family or a member of the small inducible gene (SIG) family that plays a role in the recruitment of monocytes to sites of injury and infection. These terms can also refer to C—C motif chemokine molecules, whose activity include, for example, macrophage recruitment, and/or proliferation and/or activation.

The term “alkyl,” as used herein unless otherwise defined, refers to a straight or branched saturated group derived from the removal of a hydrogen atom from an alkane. Representative straight chain alkyl groups include, but are not limited to, -methyl, -ethyl, -n-propyl, -n-butyl, -n-pentyl, and n-hexyl. Representative branched alkyl groups include, but are not limited to, isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

### DNIRA

In various embodiments, the present invention involves optical imaging, using various techniques that are known in the art. For example, such techniques include, but are not limited to cSLO, FAF, angiography, OCT, including three dimensional reconstructions of such. In various embodiments of the invention, exposing an eye to light comprises performing cSLO, FAF, DNIRA, angiography or OCT. In various embodiments, the imaging is DNIRA. In various embodiments, combinations of any of the above techniques may be used.

The inventor previously demonstrated that following systemic delivery of sodium iodate, patches of hypofluorescent FAF are not observed in vivo in areas of RPE loss (in the non-clinical setting, i.e., non-human eye) as would be predicted from clinical investigation (data not shown). However, by pre-labeling the RPE with a fluorescent dye, such as the near infra-red (NIR) dye indocyanine green (ICG), a technique called Delayed Near InfraRed Analysis (DNIRA), the RPE is made visible using cSLO imaging. Once labeled, areas of RPE loss become apparent as quantifiable patches of hypofluorescence similar to those observed clinically. In various embodiments, NaIO₃, FAF and DNIRA may be used together to show, by way of example, the relationship between RPE loss, macrophages, macrophage polarization, and regulation of the M1 response.

For DNIRA, a compound suitable for fluorescence detection including a near-infrared (NIR) dye, such as, ICG when given at non-toxic doses, can label the RPE and therefore make it visible when viewed using the ICG excitation/emission filters in the days or weeks thereafter. Importantly, this visualization in the days and weeks thereafter may be without re-administration of dye. Accordingly, in some aspects, a central component of the DNIRA technique lies in its timing. This is distinct from the present usage of ICG or other angiographic dyes that are viewed immediately after injection, during the transit phase, or in the immediate minutes to hours following injection, to determine the intra-vascular localization of dye and its immediate extravasation.

In some embodiments, DNIRA is used in a laboratory animal. In one embodiment, DNIRA may involve administration of a compound suitable for fluorescence detection, by way of non-limiting example, ICG (and, optionally, angiography) at about one or more days prior to administration with a toxin or other agent that causes patchy geographic areas of RPE loss (e.g. NaIO₃) and optionally followed by, at about 1 or more days (or about one week, or about one month, or about three months), an additional amount of NaIO₃ or another agent that causes expansion of the areas of patchy RPE loss. For example, the other challenge that causes geographic atrophy expansion (e.g. as an initial, or second, or third, or fourth administration) may be a modulator of cell survival, cell death, survival, autophagy, proliferation, regeneration, and the like.

In various embodiments, the DNIRA technique is used in a human patient. For example, DNIRA in a human patient may not comprise the use of a toxin. DNIRA in a human patient may comprise the evaluation of normal or disease-associated changes in the eye, using a fluorescent dye, with the excitation/emission filters in place but no angiography.

Expansion of geographic atrophy is a U.S. Food and Drug Administration (FDA) approved primary outcome for clinical trial design, and, accordingly, this invention makes possible observation of geographic atrophy, in particularly the expansion of geographic atrophy, in an animal model, thus permitting correlation between pre-clinical disease models and clinical trial design. The inability to clearly identify the geographic atrophy, or expansion of geographic atrophy, in an eye of an animal prior to the present invention has precluded direct correlation between pre-clinical studies and clinical observation. Further, in some embodiments, the present invention allows for clinical evaluation of geographic atrophy, including the expansion of geographic atrophy, in a human patient.

In some embodiments, the compound suitable for fluorescence detection is suitable for imaging with various wavelengths of fluorescence. In some embodiments, these wavelengths range from visible light to infrared, e.g., 390 nm to 1 mm, including, for example, blue light, white light, and near-infrared. In some embodiments, the dye is a near-infrared dye. In some embodiments, the dye is ICG.

In some embodiments, DNIRA is performed (and/or delayed near infrared fluorescence (DNIRF) is observed) at about 1 day, or about 2 days, or about 3 days, or about 4 days, or about 5 days, or about 6 days, or about 7 days, or about 10 days, or about 14 days, or about 21 day after the administration. In some embodiments, the DNIRA is performed at least 1 day after the administration, or at least 2 days, or at least 3 days, or at least 4 days, or at least 5 days, or at least 6 days, or at least 7 days, or at least 10 days, or at least 14 days, or at least 21 days after the administration. In other embodiments, the DNIRA is performed at least about 24 hours, or at least about 7 days, or at least about 30 days, or at least 60 days, or at least 90 days after administering. In other embodiments, the DNIRA is performed at least about 2 months, or about 3 months, or about 4 months, or about 5 months, or at a maximum about 6 months after administering. In some embodiments, the DNIRA is not performed during the transit stage (i.e. the first passage of dye as it flows through the ocular blood vessels and into the ocular tissue) or minutes thereafter.

In some embodiments, the visualization is effected using a cSLO. In some embodiments, the visualization is effected using white light and appropriate filters. In some embodiments, the ICG excitation/emission filters are 795 nm (excitation)/810 nm (emission) filters.

The RPE is a critical epithelial monolayer that serves a “nurse-cell” function for an eye's specialized photoreceptors, the rods and cones. Blinding eye diseases, such as, for example, AMD and RPD, are, without wishing to be bound by theory, causally linked in part to abnormalities of the RPE.

DNIRA makes it possible to clearly identify the RPE layer in vivo in an eye of an animal. Further, the leading technique used to detect the RPE in the human eye, FAF, is ineffective or poorly effective in the rodent eye (by way of non-limiting example), possibly owing to a relative paucity of fluorophores such as lipofuscin. FAF imaging in the human eye is performed using the blue spectrum of non-coherent light in the presence of stimulation/emission filters, or coherent blue light, and can identify areas of absent RPE (e.g. hypo-fluorescent signal) or abnormal RPE (e.g. hyper-fluorescent signal). The inability to clearly identify the RPE in an eye of an animal, in the absence of DNIRA, has precluded direct correlation between pre-clinical studies and clinical observation.

Accordingly, in various aspects of the present invention, methods to make visible the RPE layer, such as, for example, DNIRA, in an eye of an animal for pre-clinical investigation of ocular disorders are provided.

Further, as described herein, DNIRA, or variations thereof, allow for visualization of fluorescent immune cells in the eyes of an animal. In some embodiments DNIRA may optionally not comprise toxin administration. In some embodiments, DNIRA is performed in a human patient and a toxin is not applied.

Further, as described herein, DNIRA, or variations thereof, allow for visualization of fluorescent immune cells in the eyes of human subject. In some embodiments with a human subject DNIRA may not comprise toxin administration.

In some embodiments, DNIRA is used in the identification of an agent that is effective for treating an ocular disorder.

In some embodiments, DNIRA is used as a method to evaluate a patient that has, or may have, an ocular disorder (including, without limitation AMD and RPD). In some embodiments, DNIRA is a surrogate biomarker for diagnosis and/or prognosis and/or progression of an ocular disorder (including, without limitation AMD and RPD). For example, DNIRA may be used to identify patterns, including lacy, reticular or leopard-like pattern of alternating hyper- and hypo-fluorescent DNIRA that is not seen in other imaging modalities, that are indicative of a ocular disease state (without limitation AMD and RPD). DNIRA may also be used to identify, and quantify, areas of hyper- and hypo-fluorescent DNIRA.

In various embodiments, DNIRA is used to identify hypofluorescent features of an eye. For instance, these areas appear black when imaged and therefore allow for easy quantitation (in contrast to ICG imaging, or in contrast to hyperfluorescent signal, which is grey-scale rather than black/white). Detection of hypofluorescent DNIRA, in some embodiments, is predictive of damaged or dead RPE. For example, hypofluorescent DNIRA may indicate one or more of an absence of RPE, abnormal/unhealthy RPE (which is unable to uptake ICG dye), RPE that does not lie in contact with Bruch's Membrane (and so are no longer in a position to take up ICG dye from the choroidal vasculature), and the presence of lipid that could be located either between the RPE and BM (thus blocking ICG uptake), or could be internal to the RPE (thus blocking the RPE signal).

In various embodiments, DNIRA is used to identify hyperfluorescent features of an eye. For instance, these areas appear light when imaged and therefore allow for easy quantitation. Detection of hyperfluorescent DNIRA, in some embodiments, is predictive of macrophages, including M1 and/or M2 macrophages

In various embodiments, DNIRA is used biomarker for diagnosis of an ocular disease state (without limitation AMD and RPD) and prompts further evaluation and/or treatment with one of more agents, including without limitation those described herein.

In various embodiments, DNIRA is used as a biomarker for prognosis of an ocular disease state (without limitation AMD and RPD) and prompts further evaluation and/or treatment with one of more agents, including without limitation those described herein.

In various embodiments, DNIRA is used to improve identification of suitable patients for study recruitment and to evaluate progression of disease. In various embodiments, DNIRA is used to monitor disease progression.

In various embodiments, DNIRA is used as a companion diagnostic to any of the agents described herein. In various embodiments, DNIRA is used to evaluate patient response to any of the agents described herein (including evaluating the effectiveness of any of the agents described herein and/or the likelihood of response to any of the agents described herein). In various aspects, the present invention further relates to the use of DNIRA as entrance inclusion or exclusion criteria, or endpoint analysis for clinical trial design.

### Ocular Disorders

An ocular disorder refers to one of various ophthalmic diseases and includes diseases of the eye and the ocular adnexa. In some embodiments, the ocular disorder is, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis, wet AMD, dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR).

In various embodiments, an ocular disorder is evaluated and/or identifiable using DNIRA or a technique known in the art. Illustrative techniques include: cSLO, FAF, OCT (including with cross-sectional, three-dimensional and en face viewing), SD-OCT (with cross-sectional, three-dimensional and en face viewing), angiography, or other imaging modalities including other wavelengths of fluorescence. In some embodiments, these wavelengths range from blue to infrared, e.g., 390 nm to 1 mm, including, for example, blue light, white light, red-free, near infra-red, infrared.

In various embodiments, an ocular disorder is evaluated and/or identifiable by patterns of FAF within patches of RPE damage or loss or outer retinal loss. In some embodiments, the patterns are one or more of curvilinear, ribbon-like, reticular, oval, circular, scalloped, halo, and target-like lesions.

In various embodiments, an ocular disorder evaluated and/or identifiable by patterns of FAF within a border of patches of RPE damage or loss or outer retinal loss. In some embodiments, the patterns are one or more of curvilinear, ribbon-like, reticular, oval, circular, scalloped, halo, and target-like lesions.

In various other embodiments, an ocular disorder is evaluated and/or identifiable by cross-sectional patterns or transverse patterns. In some embodiments, the patterns are observed with OCT. In some embodiments, the patterns are RPE and/or outer retinal loss or mounds, triangles, peaks or spikes found in the sub-retinal space.

In other embodiments, an ocular disorder is evaluated and/or identifiable by the presence of areas of hyper-fluorescent FAF in an eye of the subject and/or the presence of one or more areas of abnormally fluorescent FAF in an eye of the subject and/or by changes in one or more of blue spectrum fundus imaging, white-light fundus imaging, red-free fundus imaging, and OCT in an eye of the subject and/or by an increase (e.g. a transient increase) in permeability across the subject's epithelial barrier between a choroid and a retina relative to an undiseased state and/or by a deformation of the outer retina, and/or deformation of the mid-retinal vasculature across the subject's epithelial barrier between a choroid and a retina relative to an undiseased state and/or by the presence of phagocytic immune cells across the subject's RPE relative to an undiseased state. Illustrative changes include differences in an imaging pattern in the same of different subject and/or animal at two different time points and/or experimental or clinical conditions. For example, changes can encompass changes in imaging data between two evaluations of the same subject and/or animal and/or changes in imaging data between a first subject and/or animal and the imaging data of a second subject and/or animal.

In still other embodiments, an ocular disorder is evaluated and/or identifiable by the presence of one or more areas of distinct patterns of retinal imaging in the eye of a subject, wherein the retinal imaging is one or more of white light, red-free light, blue light, FAF, near infra-red (NIR), infra-red (IR), angiography, and DNIRA and/or the presence of one or more areas of abnormally-fluorescent FAF in the eye of a subject and/or an increase (e.g. a transient increase) in permeability across the subject's epithelial barrier between a choroid and a retina relative to an undiseased state and/or a presence of phagocytic immune cells across the subject's RPE relative to an undiseased state.

In some embodiments, the ocular disorder is a diabetic eye disease, by way non-limiting example, diabetic retinopathy and DME. In some embodiments, the diabetic eye disease is found in a patient with type 1 or type 2 diabetes. In various embodiments, a diabetic eye disease patient to be treated may have a blood glucose level that exceeds normal (about 70-130 milligrams per deciliter or mg/dL before meals, and less than 180 mg/dL one to two hours after a meal).

In some embodiments, the diabetic eye disease is diabetic retinopathy in one or more of the following stages: Mild Nonproliferative Retinopathy (in which microaneurysms may occur); Moderate Nonproliferative Retinopathy (in which some blood vessels that nourish the retina may be blocked); and Severe Nonproliferative Retinopathy (in which more blood vessels may be blocked, depriving several areas of the retina with their blood supply); and Proliferative Retinopathy (in which signals sent by the retina for nourishment trigger the growth of new blood vessels.

In some embodiments, the present methods for treatment or prevention of an ocular disorder (e.g. a diabetic eye disease (for instance, diabetic retinopathy and DME), Vogt-Kayanagi-disorder (e.g. a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis) further comprise performing laser surgery (e.g. scatter laser treatment, focal laser treatment, photocoagulation) and/or vitrectomy (e.g. in which blood is removed from the center of a patient's eye) and/or administering a steroid and/or administering an anti-VEGF agent such as Ranibizumb (LUCENTIS), Bevizumab (AVASTIN) or Aflibercept (EYLEA).

In some embodiments, the diabetic eye disease is a cataract or glaucoma (e.g., neovascular glaucoma).

In various embodiments, the ocular disorder is AMD. AMD is the leading cause of blindness in the developed world. Early AMD is characterized by the accumulation of drusen, the hallmark of disease, while geographic atrophy (GA) and choroidal neovascularization (CNVM) are the blinding complications of late non-exudative (so-called “dry” or atrophic) and exudative (so-called “wet” or neovascular) disease, respectively. Anti-VEGF therapy has revolutionized treatment of wet AMD, but represents just 12-15% of all AMD cases. Though vitamin supplementation can slow the rates of progression no treatments exist for dry AMD.

In various embodiments, the ocular disorder is early non-exudative or “dry” AMD. Early non-exudative or “dry” AMD can be characterized by drusen accumulation and associated features such as pigmentation change, or RPE detachments. Late, or advanced dry AMD can be characterized by patchy atrophy of the RPE and overlying photoreceptors. These patches are visualized clinically as so-called “window defects” and are known as areas of “geographic atrophy.”

In various embodiments, the ocular disorder is wet AMD. Wet AMD is characterized by the growth of new blood vessels beneath the retina or RPE which can bleed and leak fluid, resulting in a rapid and often severe loss of central vision in the majority cases. This loss of central vision adversely affects one's everyday life by impairing the ability to read, drive and recognize faces. In some cases, the macular degeneration progresses from the dry form to the wet form.

In one embodiment, the methods pertain to treatment of exudative or wet AMD and dry AMD simultaneously in the same patient.

In one embodiment, the ocular disorder is associated with choroidal neovascularization (CNVM).

In some embodiments, AMD is detected or evaluated using FAF, an in vivo imaging method for the spatial mapping (e.g. two-dimensional, en face, and the like) of endogenous, naturally or pathologically occurring fluorophores of the ocular fundus. In some embodiments, without wishing to be bound by theory, FAF imaging allows visualization of the RPE in vivo and can help to better understand its metabolic alterations in the pathogenesis of chorioretinal disorders and retinal pigment epitheliopathies. FAF is well known in the art (see, e.g., Bellman et al. Br J Ophthalmol 2003 87: 1381-1386, the contents of which are hereby incorporated by reference).

In some embodiments, AMD and/or RPE damage or loss may also be visualized by other wavelengths of light, including white light, blue light, near infra-red, infra-red, and be visible as a “window defect” through which the choroid can be viewed. In some embodiments, AMD and/or RPE damage or loss may also be visualized by cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared).

In some embodiments, in late dry AMD, areas of geographic atrophy are clearly visualized as areas of hypofluorescent, or dark, FAF. In some embodiments, these represent areas where RPE is lost.

In some embodiments, dry AMD is identifiable by the presence of areas of hyper-fluorescent FAF in an eye of the subject. In some embodiments, the dry AMD identifiable by the presence of areas of hyper-fluorescent FAF is progressing early or late dry AMD. In some embodiments, hyper-fluorescent FAF refers to an increased fluorescence that may be caused by an enhanced visualization of a normal density of lipofuscin or lipofuscin-like materials or an increase in the lipofuscin content of the tissues. Lipofuscin comprises a mix of proteins, with or without lipid, the components of which fluoresce under blue-light illumination of the appropriate wavelengths. Lipofuscin-like fluorescence may also occur in this spectrum, and could be due to molecules other than lipofuscin and its constituents.

Lipofuscin-like fluorescence may occur in RPE cells, and cells other than RPE cells such as, for example, cells of the immune system (e.g. phagocytic immune cells).

In other embodiments, the AMD is identifiable by the presence of abnormal patterns of FAF in an eye of the subject. In some embodiments, abnormal fluorescent FAF refers to deviation from the normal fluorescent FAF pattern observed in a subject's eye. In normal FAF, using the cSLO or modified fundus cameras, the optic nerve head is dark (black) due to the absence of RPE (and hence no lipofuscin) and the blood vessels are also dark because they block fluorescence from the underlying RPE monolayer. In the central macular area, the FAF signal is reduced by absorption of blue light by luteal pigment. These characteristics of normal blue-light FAF may be considered when evaluating for the presence of abnormal fluorescence.

In some embodiments, hyperfluorescent FAF associated with AMD and other blinding diseases, may show two-dimensional spatial patterns, which may be complex. In some studies, these patterns of hyperfluorescent FAF correlate with rates of disease progression from early to late (either neovascular or atrophic) disease. These patterns are understood in the art (see, e.g., Schmitz-Valckenberg et al. Survey of Ophthalmology. 2009 January-February; 54(1):96-117; Bindewald et al. British Journal of Ophthalmology. 2005 July; 89(7):874-8; Holz et al. Am. J Ophthalmology. 2007 March; 143(3):463-72, the contents of which are hereby incorporated by reference in their entireties).

In some embodiments, the dry AMD is early stage AMD, or is late stage, atrophic dry AMD.

In some embodiments, dry AMD is in its late stage and characterizable by the presence of areas of hyper-fluorescent or abnormally-fluorescent FAF in areas bordering and/or adjacent to (in the so-called junctional zone) pre-existent geographic atrophy.

In some embodiments, dry AMD is in its late stage and characterizable by the presence of areas of hyper-fluorescent or abnormally-fluorescent FAF in the absence of pre-existent geographic atrophy. In these embodiments, without wishing to be bound by theory, it may predict future loss of the RPE.

In some embodiments, dry AMD in both early and late stage is characterizable by the presence of immune cells (e.g. phagocytic immune cells). The presence of immune cells can be surmised from post-enucleation or post-mortem ocular samples. As described herein, in some embodiments, the presence of immune cells is assessed using DNIRA.

In some embodiments, disorders associate with imaging (FAF) patterns such as “diffuse trickling” may be evaluated, treated or prevented using methods disclosed herein. Diffuse trickling patterns are known in the art (see, e.g., Schmitz-Valckenberg et al. Survey of Ophthalmology. 2009 January-February; 54(1):96-117; Bindewald et al. British Journal of Ophthalmology. 2005 July; 89(7):874-8; Holz et al. Am. J Ophthalmology. 2007 March; 143(3):463-72, the contents of which are hereby incorporated by reference in their entireties).

In some embodiments, the dry AMD is identifiable by an increase (e.g. a transient increase) in permeability across the subject's epithelial barrier between a choroid and a retina, relative to an undiseased state. This is distinct from vascular (endothelial) permeability. For example, this may be seen using angiography.

In some embodiments, DNIRA is used to identify and/or diagnoses any of the ocular disorder described herein. In some embodiments, RPE toxicity, RPE loss, and the dry AMD are identifiable using DNIRA, e.g. with sodium iodate (NaIO₃) or in the presence of disease. Areas analogous to geographic atrophy can be detected by, for example, tissue analysis (for example, by observing the loss of an RPE cell marker such as RPE65 and/or the loss of binucleate cell staining), and/or in vivo, using DNIRA, of NaIO₃_treated-eyes. In some embodiments, RPE toxicity, RPE loss and the dry AMD are identifiable using FAF which shows a hyperfluorescent FAF signal, which may be complex, within the area of imminent tissue loss and/or at its margins. This complex hyperfluorescent FAF signal develops, in the days or weeks after NaIO₃ treatment, into a pattern of FAF, which can be complex and can include, but is not limited to ribbon-like, curvilinear, pisciform, scalloped, interlacing, branching, or reticular hyperfluorescence. Such patterns mimic those found in clinical dry AMD. Such FAF may also be hypofluorescent.

Further, using angiography alone, or as part of DNIRA, angiography performed immediately prior to, or coincident with, the emergence of the areas of altered FAF may demonstrate an increase (including a transient increase) in permeability across the epithelial barrier between choroid and retina (such as, for example, observed by leakage of dyes that may be injected prior to imaging; such dyes include ICG). This barrier normally includes the inner choroid, Bruch's Membrane, the RPE cells, and, possibly, the configuration of the outer photoreceptors in conjunction with the RPE and the outer limiting membrane. Without wishing to be bound by theory, a transient breakdown of this specialized epithelial barrier may underlie a sequence of events thereafter including folding, undulation, or deformation of the outer retina, photoreceptor layer, and/or movement/migration of inflammatory cells from the choroid to the subretinal space. However, in some embodiments, this phase may be transient and resolved, or subclinical in its extent.

In some embodiments, the dry AMD is identifiable by a presence (e.g. an influx) of immune (e.g. phagocytic immune cells or innate immune cells) cells across the subject's retinal pigment epithelium (RPE) relative to an undiseased state. When there is movement/migration of inflammatory cells from the choroid to the subretinal space, or inner retina to outer retina or subretinal space, such cells may be identified in enucleated eyes or excised tissue by staining methods known in the art. In the NaIO₃ model, IbaI staining may be used to detect activated cells of the immune system. Further, the presence of these cells may be confirmed by comparison with, for example, NaIO₃ preparations in which monocyte/macrophage depletion have been undertaken. Such depletion is known in the art and may be achieved by treatment with, for example, gadolinium chloride (GAD) or clodronate. In some embodiments, the presence (e.g. an influx) of immune cells is measured and/or determined, in vivo, by use of DNIRA. Prior to the present invention, such in vivo visualization was not possibly in the clinical setting.

Further, macrophages, an example of an immune cell that may be identified in the cells of a subject, may be classified by subsets: classically (M1) or alternatively (M2) activated macrophages (see, e.g., Laskin, Chem Res Toxicol. 2009 Aug. 17; 22(8): 1376-1385, the contents of which are hereby incorporated by reference in their entireties). Without wishing to be bound by theory, M1 macrophages are activated by standard mechanisms, such as IFNγ, LPS, and TNFα, while M2 macrophages are activated by alternative mechanisms, such as IL-4, IL-13, IL-10, and TGFβ. Without wishing to be bound by theory, M1 macrophages display a cytotoxic, proinflammatory phenotype, while M2 macrophages, suppress some aspects of immune and inflammatory responses and participate in wound repair and angiogenesis. In some embodiments, the invention comprises the inhibition, modulation or polarization of one or more of M1 and M2 macrophages.

In some embodiments, the dry AMD is identifiable by the changes in one or more of blue spectrum fundus imaging, white-light fundus imaging, red-free fundus imaging, and OCT in an eye of the subject. In some embodiments, the dry AMD is identifiable by the changes in one or more of cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared). Illustrative changes include differences in an imaging pattern in the same of different subject and/or animal at two different time points and/or experimental or clinical conditions. For example, changes can encompass changes in imaging data between two evaluations of the same subject and/or animal and/or changes in imaging data between a first subject and/or animal and the imaging data of a second subject and/or animal.

In some embodiments, the AMD is identifiable by OCT. In some embodiments, cross-sectional images show the presence of shallow mounds, or pyramidal or spike-like signals, in the subretinal space. Three-dimensional or en face OCT imaging reveals ribbon-like or curvilinear, oval, circular, halo or target-like signals. In some embodiments, cross-sectional patterns or transverse patterns are observed and the cross-sectional or transverse patterns comprise RPE and/or outer retinal loss (atrophy) or mounds, triangles, peaks or spikes found in the sub-retinal space. In various embodiments, these features are indicative of AMD.

The invention further provides methods for treatment or prevention of RPD disease, also known as, but not limited to, the following: “reticular drusenoid disease,” “pseudoreticular drusen,” and “drusenoid macular disease,” or “disease characterized by the presence of subretinal drusenoid deposits.” Not wishing to be bound by theory, RPD may be a subtype of, or may be related to AMD. Both AMD and RPD can lead to the blinding complications of choroidal neovascularization or geographic atrophy.

In some embodiments, the RPD disease is that in which pseudodrusen material is reduced or eradicated or the accumulation and/or expansion of which is slowed upon treatment. In some embodiments, the invention provides a method for treating RPD disease in which the pseudodrusen are reduced or eradicated upon treatment in the foveal area and/or perifoveal area and/or juxtafoveal area of a subject's eye.

In some embodiments, the invention further provides methods for treatment or prevention of RPD disease in which the rates of progression from early to late disease are reduced. Late disease can include, for example, choroidal neovascularization or geographic atrophy

In some embodiments, the invention further provides methods for treatment or prevention of RPD disease in which the rates of expansion of geographic atrophy are reduced.

In some embodiments, the RPD disease is identifiable by FAF or other imaging modalities including other wavelengths of fluorescence. In some embodiments, these wavelengths range from blue to infrared, e.g., 390 nm to 1 mm, including, for example, blue light, white light, near-infrared, and infra-red.

In some embodiments, the RPD disease is identifiable by the presence of areas of hyper- and/or abnormal-fluorescent FAF or other imaging modalities including other wavelengths of fluorescence. In some embodiments, these wavelengths range from blue to infrared e.g., 390 nm to 1 mm, including, for example, blue light, white light, near-infrared and infra-red. In some embodiments, the RPD disease is identifiable by using DNIRA.

In some embodiments, the RPD disease is identifiable using at least one of, or at least two of, or at least three of, or at least four of white light, blue light, FAF, and near infrared and infrared. In a specific embodiment, the RPD disease is identifiable by blue light.

In some embodiments, the RPD disease is identifiable by OCT. In some embodiments, cross-sectional images show the presence of shallow mounds, or pyramidal or spike-like signals, in the subretinal space. Three-dimensional or en face OCT imaging reveals ribbon-like or curvilinear, oval, circular, halo or target-like signals.

In some embodiments, the RPD is identifiable by an increase (e.g. a transient increase) in permeability across the subject's epithelial barrier between a choroid and a retina, relative to an undiseased state. This is distinct from vascular (endothelial) permeability. For example, using DNIRA, which comprises, for example, a sodium iodate (NaIO₃) model of RPE toxicity, geographic areas of RPE damage or loss can be formed that can be detected by, for example, tissue analysis (for example, by observing the loss of an RPE cell marker such as RPE65 and/or the loss of binucleate cell staining). Further FAF imaging of NaIO₃-treated RPE shows a hyperfluorescent FAF signal, which may be complex, within the area of imminent tissue loss and/or at its margins. This hyperfluorescent FAF signal develops, in the days or weeks after NaIO₃ treatment, into a pattern, which can be complex, of FAF that includes, but is not limited to ribbon-like, curvilinear, pisciform, scalloped, interlacing, branching, or reticular hyperfluorescence. Such patterns mimic those found in clinical RPD.

Further, in DNIRA, angiography performed immediately prior to, or coincident with, the emergence of the areas of altered FAF demonstrates an increase (including a transient increase) in permeability across the epithelial barrier between choroid and retina (such as observed by leakage of dyes that may be injected prior to imaging; such dyes include ICG). This barrier normally includes the inner choroid, Bruch's Membrane, the RPE cells, and, possibly, the configuration of the outer photoreceptors in conjunction with the RPE and the outer limiting membrane. Without wishing to be bound by theory, a transient breakdown of this specialized epithelial barrier may be permissive for the sequence of events thereafter including folding, undulation, or deformation of the photoreceptor layer. However, in some embodiments, this phase may be transient and resolved, or subclinical in its extent.

In some embodiments, the RPD is identifiable by a presence (e.g. an influx) of immune cells (e.g. phagocytic immune cells) across the subject's retinal pigment epithelium (RPE) relative to an undiseased state. In some embodiments, presence (e.g. an influx) of immune cells is detected and/or measured with DNIRA. When there is movement/migration of inflammatory cells from the choroid to the subretinal space, or inner retina to outer retina or subretinal space, such cells may be identified by staining, as is known in the art. For example, using a NaIO₃ model, IbaI staining may be used to detect activated phagocytic cells of the immune system. Further, the presence of these cells may be confirmed by comparison with NaIO₃ preparations in which monocyte/macrophage depletion has been undertaken. Such depletion is known in the art and may be achieved by treatment with, for example, gadolinium chloride (GAD) or clodronate. Further, macrophages, an example of a phagocytic immune cell, that are identified in the cells of a subject, may be classified by subsets: classically (M1) or alternatively (M2) activated macrophages (see, e.g., Laskin, Chem Res Toxicol. 2009 Aug. 17; 22(8): 1376-1385, the contents of which are hereby incorporated by reference in their entireties). Without wishing to be bound by theory, M1 macrophages are activated by standard mechanisms, such as IFNγ, LPS, and TNFα, while M2 macrophages are activated by alternative mechanisms, such as IL-4, IL-13, IL-10, and TGF. Without wishing to be bound by theory, M1 macrophages display a cytotoxic, proinflammatory phenotype, while M2 macrophages, suppress some aspects of immune and inflammatory responses and participate in wound repair and angiogenesis. For example, the present invention, in some embodiments, comprises the inhibition, modulation or polarization of one or more of M1 and M2 macrophages.

In some embodiments, the RPD disease is identifiable by the changes in one or more of blue spectrum fundus imaging, white-light fundus imaging, red-free fundus imaging, and OCT in an eye of the subject. In some embodiments, the RPD disease is identifiable by the changes in one or more of cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared). Illustrative changes include differences in an imaging pattern in the same of different subject and/or animal at two different time points and/or experimental or clinical conditions. For example, changes can encompass changes in imaging data between two evaluations of the same subject and/or animal and/or changes in imaging data between a first subject and/or animal and the imaging data of a second subject and/or animal.

In another embodiment, the ocular disorder is LORDs (late onset retinal degeneration) or retinal degeneration associated with clqTNF5 deficiency or its corresponding gene mutation, or another maculopathy, including, but not limited to, Stargart disease, pattern dystrophy, as well as retinitis pigmentosa (RP) and related diseases. In one embodiment, the maculopathy is inherited.

In other embodiments, the blinding eye disease is an idiopathic disorder that may, without wishing to be bound by theory, be characterized by retinal inflammation, with or without accompanying macular degeneration, including, but not limited to, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), and diffuse subretinal fibrosis (DSF)).

In other embodiments, the ocular disorder is central serous retinopathy (CSR). CSR is a fluid detachment of macula layers from their supporting tissue. CSR is often characterizable by the leak and accumulation of fluid into the subretinal or sub-RPE space. Without wishing to be bound by theory, the leak and accumulation of fluid may occur because of small breaks in the RPE.

In some embodiments, one or more of the ocular disorder are identifiable by the presence of areas of hyper- and/or abnormal-fluorescent FAF, or other wavelengths of light from 350 nm to 1,000 nm. In some embodiments, one or more of the ocular disorders are identifiable using DNIRA.

In some embodiments, one or more of the ocular disorders are identifiable by, for example, cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared).

In some embodiments, the ocular disorder may be of a certain stage or progression. In some embodiments, the ocular disorder may be incipient, emerging, quiescent, advancing or active.

In addition to treating pre-existing ocular disorders, the present invention comprises prophylactic methods in order to prevent or slow the onset of these disorders. In prophylactic applications, an agent can be administered to a subject susceptible to or otherwise at risk of a particular ocular disorder. Such susceptibility may be determined by, for example, familial predisposition, genetic testing, risk factor analysis, blood or other cytokine or biomarker levels, and ocular examination, which can include multi-modal analysis such as FAF, blue light, white light, red-free, near infra-red, infrared, DNIRA, etc. Such susceptibility may also be determined by, for example, detection by OCT, with cross-sectional, three-dimensional and en face viewing.

In some embodiments, the ocular disorder is one or more ailments listed in the International Statistical Classification of Diseases and Related Health Problems, or ICD-10 in the following sections (which are incorporated herein by reference): H30 chorioretinal inflammation, H31 other disorders of choroid, H32 chorioretinal disorders in diseases classified elsewhere, H33 retinal detachments and breaks, H34 retinal vascular occlusion, H35 other retinal disorders, and H36 retinal disorders in diseases classified elsewhere.

In addition to treating defined, known ocular disorders, the present invention comprises particular patterns of in vivo imaging using light at wavelengths ranging from 300 to 1,000 nm, including white light, blue light, FAF, infra-red, near infra-red, DNIRA or by OCT, with cross-sectional, three-dimensional and en face viewing. In applications against particular patterns of in vivo imaging, an agent can be administered to a subject with, susceptible to, or otherwise at risk of a particular blinding disease. Such diagnosis or susceptibility may be determined by, for example, ophthalmic examination, familial predisposition, genetic testing, risk factor analysis, and blood or other cytokine or biomarker levels, which can include multi-modal analysis such as FAF, blue light, white light, red-free, near infra-red, infrared, DNIRA, etc. Such susceptibility may also be determined by, for example, detection by OCT, with cross-sectional, three-dimensional and en face viewing.

### Agents of the Invention

In various aspects, any compound or agent described herein is an agent of the invention.

In various aspects, an agent of the invention is one or more compounds or genera described in one or more of the following documents, the contents of which are hereby incorporated by reference in their entireties: International Patent Publication No. WO/2009/083436 (and U.S. Pat. Nos. 8,399,477 and 8,871,948); International Patent Publication No. WO/2009/109616 (and U.S. Pat. No. 8,461,194); International Patent Publication No. WO/2009/109618 (and U.S. Pat. Nos. 7,919,518 and 8,283,348); International Patent Publication No. WO/2009/109654 (and U.S. Pat. Nos. 8,314,099 and 8,569,297); International Patent Publication No. WO/2009/109613 (and U.S. Pat. No. 8,835,481); and International Patent Publication No. WO/2011/015501 (and U.S. Pat. No. 8,354,544).

In various aspects, the present invention provides for the identification and use of a candidate compound and/or test compound. In embodiments providing for identification and use of a candidate compound and/or test compound, the candidate compound and/or test compound may be chemical, molecule, compound, biologic (e.g. an antibody or peptide), drug, pro-drug, cellular therapy, low molecular weight synthetic compound, or a small molecule drug. In some embodiments, the candidate compound and/or test compound is selected from a library of compounds known in the art. In some embodiments, the candidate compound is useful for treating an ocular disorder, preventing a binding eye disease, or reducing the rate of pathogenesis of a ocular disorder.

In various aspects, the present invention provides for a method for treating or preventing any of the diseases or disorders described herein, including diabetic blinding eye diseases, dry AMD and/or RPD. In some embodiments, a compound of Formulae I-IV, or a pharmaceutically acceptable salt thereof, is also administered with an additional therapeutic agent. In various embodiments, the additional therapeutic agent may be used in a combination therapy with the agents effective for treating an ocular disorder described herein. In various embodiments, the agents effective for treating an ocular disorder described herein is administered to a patient undergoing treatment with one or more additional therapeutic agents. Additional therapeutic agents, include, for example, one or more of an anti-VEGF agent, an ACE inhibitor, a PPAR-gamma agonist or partial agonist, a renin inhibitor, a steroid, and an agent that modulates autophagy, as well as a semapimod, a MIF inhibitor, a CCR2 inhibitor, CKR-2B, a 2-thioimidazole, CAS 445479-97-0, CCX140, clodronate, a clodonate-liposome preparation or gadolinium chloride.

In various aspects, the present invention provides for identifying an ocular disorder subject who is more likely than not to respond to treatment with an agent (e.g. an “agent of the invention”) and/or determining whether an ocular disorder in a subject is responsive to treatment with an agent.

In some embodiments, an agent of the invention is a compound of Formula I:

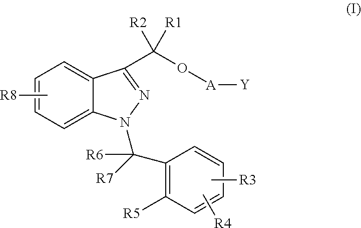

or a pharmaceutically acceptable salt thereof, wherein:

A may be a bond a, —X₁— or —X₁—O—X₂—, in which

X₁ and X₂, which may be identical or different from each other, may be an alkyl group having from 1 to 5 carbon atoms, optionally substituted with one or more alkyl groups having from 1 to 5 carbon atoms or one or more alkoxy groups having from 1 to 3 carbon atoms,

Y is H when A is a bond a, or Y may be H, —OH, or —N(R₁₁)(R₁₂), when A is —X₁— or —X₁—O—X₂—, in which

R₁₁ may be hydrogen, an alkyl group having from 1 to 5 carbon atoms, or R₁₁ together with R₁₂ forms a 4- to 7-membered heterocycle,

R₁₂ may be hydrogen, an alkyl group having from 1 to 5 carbon atoms, or R₁₂ together with R₁ forms a 4- to 7-membered heterocycle,

R₁ and R₂, which may be identical or different from each other, may be hydrogen, an alkyl group having from 1 to 5 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms,

R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms.

In certain embodiments, the agent of the invention is a compound of Formula I with the proviso that when A is a σ bond, and Y, R₁, R₂, R₆, and R₇ are hydrogen atoms,

if R₈ is a hydrogen atom, then the group linked to the nitrogen atom in the 1-position of the indazole ring is different from a benzyl group, a 4-chlorobenzyl group, or a 2-4-dichlorobenzyl group,

if R₈ is a fluorine atom in the 5-position of the indazole ring, then the group linked to the nitrogen atom in the 1-position of the indazole ring is different from 5-chloro-2-methoxybenzyl group, and

if R₈ is a trifluoromethyl group in the 6-position of the indazole ring, then the group linked to the nitrogen atom in the 1-position of the indazole ring is different from a 2-4-dichlorobenzyl group.

In certain embodiments, X₁ and X₂ are, independently of one another, an alkyl group having from 1 to 4 carbon atoms, optionally substituted with one or more alkyl groups having from 1 to 3 carbon atoms or one or more alkoxy groups having 1 or 2 carbon atoms.

In certain embodiments, X₁ is selected from a CH₂ group, a CH₂CH₂ group, a C(CH₃)₂ group, or a C(CH₃)₂CH₂ group. In certain embodiments, X₂ is selected from a CH₂ group, a CH₂CH₂ group or a CH₂CH₂CH₂ group.

In certain embodiments, residue A is represented by a σ bond, a CH₂CH₂ group, a CH₂CH₂CH₂ group, a C(CH₃)₂CH₂ group, a CH₂CH₂OCH₂ group, a CH₂CH₂OCH₂CH₂ group, a C(CH₃)₂CH₂OCH₂ group, or a C(CH₃)₂CH₂OCH₂CH₂ group.

In certain embodiments, when A is a bond a, then Y is a hydrogen atom, while when A is —X₁— or —X₁—O—X₂— then Y may be hydrogen, —OH, or —N(R₁₁)(R₁₂).

In certain embodiments, R₁₁ is represented by a hydrogen atom, an alkyl group having from 1 to 3 carbon atoms, or R₁₁ together with R₁₂ forms a 5- or 6-membered heterocycle.

In certain embodiments, R₁₂ is represented by a hydrogen atom, an alkyl group having from 1 to 3 carbon atoms, or R₁₂ together with R₁ forms a 5- or 6-membered heterocycle.

In certain embodiments, R₁ and R₂, which may be identical or different, are represented by a hydrogen atom or an alkyl group having from 1 to 3 carbon atoms.

In certain embodiments, R₃, R₄ and R₈, which may be identical or different from each other, are represented by a hydrogen atom, an alkyl group having from 1 to 3 carbon atoms, an alkoxy group having 1 or 2 carbon atoms, a Br, Cl or F atom, an OH group, a nitro group, a trifluoromethyl group or the group N(R′)(R″), —N(R′)COR″; —CN, —CONR′R″, —SO₂NR′R″, —SO₂R′, with R′ and R″, which may be identical or different, represented by a hydrogen atom and an alkyl group having from 1 to 3 carbon atoms.

In certain embodiments, R₅ is represented by a hydrogen atom, an alkyl group having from 1 to 3 carbon atoms, an alkoxy group having 1 or 2 carbon atoms, a halogen atom, an OH group, or R₅, together with one from between R₆ and R₇, forms a ring having 5 or 6 carbon atoms.

In certain embodiments, R₆ and R₇, which may be identical or different, are represented by a hydrogen atom, an alkyl group having from 1 to 3 carbon atoms, or together form a group C═O, or one from between R₆ and R₇, together with R₅, forms a ring having 5 or 6 carbon atoms.

In certain embodiments, the compound of Formula I according to the present invention may have an asymmetric carbon atom and may then be in the form of stereoisomers and enantiomers.

In certain embodiments, an agent of the invention is a compound of Formula I:

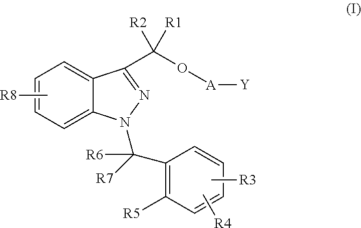

wherein:

A is a σ bond;

Y is H or —N(CH₃)₂;

R₁, R₂, and R₅ each independently is H or —CH₃;

R₃ is H, a Cl or F atom, —CH₃, or —N(CH₃)₂;

R₄ is H or a Cl atom;

R₆ and R₇ each independently is H or R⁶ and R⁷ together form a group C═O;

R₈ is H or a Cl atom at position 5 of the imidazole ring, or a pharmaceutically acceptable salt, ester, or prodrug thereof.

The agent of the invention is additionally described in, for example, WO2009/109616, U.S. Pat. No. 8,461,194, and U.S. Patent Publication No. 2013/0267704, the entire disclosures of all of which are hereby incorporated by reference.

In some embodiments, an agent of the invention is a compound of Formula II:

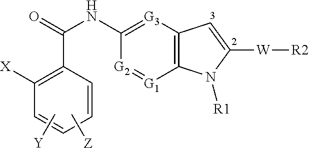

or a pharmaceutically acceptable salt thereof, wherein:

X is a halogen atom or a (C₁-C₃)alkyl, trifluoromethyl, nitro, amino, cyano, di(C₁-C₃)alkylamino, hydroxy, (C₁-C₃)alkoxy, phenyl or (C₁-C₃)alkylphenyl group;

Y and Z, which may be identical or different, are a hydrogen or halogen atom, or a (C₁-C₃)alkyl, trifluoromethyl, nitro, amino, di(C₁-C₃)alkylamino, hydroxy, (C₁-C₃)alkoxy, phenyl, COOH, (C₁-C₃)alkyl-COOH, (C₂-C₃)alkenyl-COOH, COOR, wherein R is a linear or branched (C₁-C₆)alkyl or hydroxyalkyl group, CONH₂, SO₂CH₃, SO₂NHCH₃ or NHSO₂CH₃ group;

G1, G2, and G3, which may be identical or different, are a nitrogen atom or a CH group;

R1 is a (C₁-C₆)alkyl, (C₃-C₇)cycloalkyl, (C₁-C₆)alkylOR^{I}, (CH₂)ₙNR^{II}R^{III}, (CH₂)ₙCONR^{II}R^{III}, (CH₂)ₙCOR^{I}, (CH₂)ₙCOOR^{II}, (CH₂)ₙOCOR^{I}, SO₂R^{I}, (CH₂)ₙNR^{II}SO₂R^{I}, (CH₂)ₙSO₂R^{I} group, optionally substituted with 1 to 3 hydroxy groups, wherein n is an integer from 1 to 6, R^{I} is a (C₁-C₃)alkyl, or (C₁-C₃)alkylOH group, and R^{II} and R^{III}, which may be identical or different, are a hydrogen atom or a (C₁-C₃)alkyl group;

W is a σ bond, or a (C₁-C₆)alkyl, (C₂-C₆)alkenyl, O(C₁-C₆)alkyl, O(C₂-C₆)alkenyl, C(O)NH, (CH₂)ₚCO(CH₂)_{q}, or (CH₂)ₚC(OH)(CH₂)_{q} group,

wherein p and q, which may be identical or different, are an integer from 0 to 3;

R2 is a phenyl, pyridine or (C₃-C₇)cycloalkyl group, optionally substituted with 1 to 3 substituents, which may be identical or different, represented by a L-M group, wherein L is a σ bond, or a (C₁-C₆)alkyl, (C₂-C₆)alkenyl, (C₂-C₆)alkinyl, O(C₁-C₆)alkyl, O(C₂-C₆)alkenyl, O(C₂-C₆)alkinyl group, and M is a hydrogen or halogen atom, or a OH, CF₃, NO₂, CN, COOR^{II}, SO₂NHR^{II}, CH₂CONR^{II}R^{III}, NR^{II}R^{III}, SO₂R^{IV}, NHSO₂R^{IV}, POR^{IV}R^{V}, or OPOR^{IV}R^{V} group, wherein R^{II} and R^{III}, which may be identical or different, have the meaning above, and R^{IV} and R^{V}, which may be identical or different, are a (C₁-C₃)alkyl group,

provided that

when G1, G2, and G3 are all a CH group, R₁ is a (C₁-C₆)alkyl or (C₃-C₇)cycloalkyl group, optionally substituted with 1 to 3 hydroxy groups, W is a σ bond, and the bond between the carbon atoms in the 2 and 3 position is a double bond,

R₂ is not a phenyl or pyridine group, optionally substituted with 1 to 3 substituents, which may be identical or different, selected from halogen, (C₁-C₆)alkyl optionally substituted with a hydroxy group, trifluoromethyl, nitro, amino, di(C₁-C₃)alkylamino, hydroxy, (C₁-C₃)alkoxy, COOH, COOR^{II}, SO₂CH₃, SO₂NHCH₃, NHSO₂CH₃, POR^{IV}R^{V}, OPOR^{IV}R^{V}, (C₁-C₆)alkyl-COOH, and (C₂-C₆)alkenyl-COOH,

and provided that when G1 is N, and G2 and G3 are a CH group, R₂ is not a divalent aromatic group substituted with one L-M group represented by O(C₁-C₆)alkyl, O(C₂-C₆)alkenyl, and O(C₂-C₆)alkinyl group.

In some embodiments, an agent of the invention is a compound of Formula III:

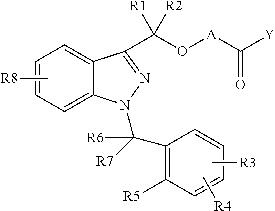

or a pharmaceutically acceptable salt thereof, wherein:

A may be —X₁ or —X₁—OC(R₉)(R₁₀)—, in which
X₁ may be an alkyl group having from 1 to 5 carbon atoms, optionally substituted with one or more alkyl groups having from 1 to 5 carbon atoms or one or more alkoxy groups having from 1 to 3 carbon atoms, and
R₉ and R₁₀, which may be identical or different from each other, may be hydrogen, an alkyl group having from 1 to 5 carbon atoms or an alkoxy group having from 1 to 3 carbon atoms,

Y may be N(R₁₁)(R₁₂), N(R₁₃)O(R₁₄), N(R₁₃)N(R₁₄)(R₁₅), N(R₁₃)—X₂—N(R₁₄)(R₁₅), N(R₁₃)—X₂—CO—X₃, in which
R₁₁ may be hydrogen, an alkyl group having from 1 to 5 carbon atoms, an alkoxy group having from 1 to 3 carbon atoms, or R₁₁ together with R₁₂ forms a 4- to 7-membered heterocycle,
R₁₂ may be hydrogen, an alkyl group having from 1 to 5 carbon atoms, an alkoxy group having from 1 to 3 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms, or R₁₂ together with R₁₁, forms a 4- to 7-membered heterocycle,
R₁₃ and R₆₅, which may be identical or different from each other, may be hydrogen, an alkyl group having from 1 to 5 carbon atoms or an alkoxy group having from 1 to 3 carbon atoms,
R₁₄ may be hydrogen, an alkyl group having from 1 to 5 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms,
X₂ may be an alkyl group having from 1 to 5 carbon atoms, optionally substituted with one or more alkyl groups having from 1 to 5 carbon atoms or one or more alkoxy groups having from 1 to 3 carbon atoms, X₃ may be OH, NH₂, NHOH or NHNH₂,

R₁ and R₂, which may be identical or different from each other, may be hydrogen, an alkyl group having from 1 to 5 carbon atoms or an alkoxy group having from 1 to 3 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R′)(R″), —N(R′)COR″, nitro and trifluoromethyl, or R₅ together with one from among R₆ and R₇ forms a ring having 5 or 6 carbon atoms; with R′ and R″, which may be identical or different from each other, represented by hydrogen and an alkyl group having from 1 to 5 carbon atoms,

R₆ and R₇, which may be identical or different from each other, may be hydrogen, an alkyl group having from 1 to 5 carbon atoms, or together form a group C═O, or one from among R₆ and R₇, together with R₅, forms a ring having 5 or 6 carbon atoms.

In some embodiments, an agent of the invention is a compound of Formula IV:

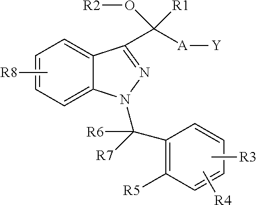

or a pharmaceutically acceptable salt thereof, wherein:

A may be —X₁— or —X—O—X₂—, in which
X₁ may be an alkyl group having from 1 to 5 carbon atoms, optionally substituted with one or more alkyl groups having from 1 to 5 carbon atoms or one or more alkoxy groups having from 1 to 3 carbon atoms, and
X₂ may be an alkyl group having from 1 to 5 carbon atoms, optionally substituted with one or more alkyl groups having from 1 to 5 carbon atoms,

Y may be hydrogen, —OH, —N(R₁₁)(R₁₂), —N(R₁₁)O(R₁₂), in which
R₁₁ may be hydrogen, an alkyl group having from 1 to 5 carbon atoms, an alkoxy group having from 1 to 3 carbon atoms, or R₁₁ together with R₁₂ forms a 4- to 7-membered heterocycle,
R₁₂ may be hydrogen, an alkyl group having from 1 to 5 carbon atoms, an alkoxy group having from 1 to 3 carbon atoms, an aryl group, a heteroaryl group, an alkylaryl group, an alkylheteroaryl group, COR′, COOR′, CON(R′)(R″) with R′ and R″, which may be identical or different from each other, represented by hydrogen and an alkyl group having from 1 to 5 carbon atoms, or R₁₂ together with R₁₁, forms a 4- to 7-membered heterocycle,

R₁ and R₂, which may be identical or different from each other, may be hydrogen, an alkyl group having from 1 to 5 carbon atoms,

R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,

R₅ may be hydrogen, an alkyl group having from 1 to 5 carbon atoms, an alkoxy group having from 1 to 3 carbon atoms, a halogen atom, —OH, —N(R′)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms,

R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms.

In still another embodiment, an agent of the invention is a modulator of macrophage polarization. Illustrative modulators of macrophage polarization include peroxisome proliferator activated receptor gamma (PPAR-g) modulators, including, for example, agonists, partial agonists, and antagonists or combined PPAR-gamma/alpha agonists.

In some embodiments, the PPAR gamma modulator is a full agonist or a partial agonist. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, rhodanine. In some embodiments, the PPAR gamma modulator is one or more of irbesartan and telmesartan. In some embodiments, the PPAR gamma modulator is a nonsteroidal anti-inflammatory drugs (NSAID, such as, for example, ibuprofen) and indoles. Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11):750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In another embodiment, an agent of the invention is semapimod (CNI-1493) as described in Bianchi, et al. (March 1995). Molecular Medicine (Cambridge, Mass.) 1 (3): 254-266, the contents of which are hereby incorporated by reference in their entireties.

In still another embodiment, an agent of the invention is a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S,R)-3-(4-hydroxyphenyl)-4,5-dihydro-5-isoxazole acetic acid methyl ester (ISO-1), isoxazoline, p425 (J. Biol. Chem., 287, 30653-30663), epoxyazadiradione, or vitamin E.

In still another embodiment, an agent of the invention is a chemokine receptor 2 (CCR2) inhibitor as described in, for example, U.S. Patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (Plos ONE 7(3): e32864, the contents of which are hereby incorporated by reference).

In various specific embodiments, an agent of the invention is one or more of CKR-2B, a 2-thioimidazole, CCR2 Antagonist (CAS 445479-97-0), and CCX140.

In yet another embodiment, an agent of the invention is a clodronate. In still another embodiment, the agent is a clodronate liposome preparation as described in Barrera et al. Arthritis and Rheumatism, 2000, 43, pp. 1951-1959, the contents of which are hereby incorporated by reference in their entireties.

In still another embodiment, an agent of the invention is a chelated or unchelated form of gadolinium, for example gadolinium chloride (GAD).

In various embodiments an agent of the invention is an anti-VEGF agent. Non limiting examples of anti-VEGF agents useful in the present methods include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VAO1 antibody, BL2 antibody, VEGF-related protein, sFLT01, sFLT02, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C (VEGF-C), human vascular endothelial growth factor-D (VEGF-D) and human vascular endothelial growth, factor-E (VEGF-E), and an antibody that binds, to an epitope of VEGF.

In one embodiment, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three dimensional structure of VEGF that is displayed, such that the epitope is exposed on the surface of the folded VEGF molecule. In one embodiment, the epitope is a linear amino acid sequence from VEGF.

In various embodiments, an agent of the invention is a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non limiting examples of renin inhibitors which are useful in the present invention include, but are not limited to: aliskiren, ditekiren, enalkiren, remikiren, terlakiren, ciprokiren and zankiren, pharmaceutically acceptable salts thereof, and mixtures thereof.

In various embodiments an agent of the invention is a steroid. In some embodiments, a steroid is a compound belonging to or related to the following illustrative families of compounds: corticosteroids, mineralocosteroids, and sex steroids (including, for example, potentially androgenic or estrogenic or anti-androgenic and anti-estrogenic molecules). Included amongst these are, by way of non-limiting example, prednisone, prednisolone, methyl-prednisolone, triamcinolone, fluocinolone, aldosterone, spironolactone, danazol (otherwise known as OPTINA), and others.

In various embodiments an agent of the invention is an agent that modulates autophagy, microautophagy, mitophagy or other forms of autophagy. In some embodiments, the candidate drug and/or compound is one or more of sirolimus, tacrolimis, rapamycin, everolimus, bafilomycin, chloroquine, hydroxychloroquine, spautin-1, metformin, perifosine, resveratrol, trichostatin, valproic acide, Z-VAD-FMK, or others known to those in the art. Without wishing to be bound by theory, agent that modulates autophagy, microautophagy, mitophagy or other forms of autophagy may alter the recycling of intra-cellular components, for example, but not limited to, cellular organelles, mitochondria, endoplasmic reticulum, lipid or others. Without further wishing to be bound by theory, this agent may or may not act through microtubule-associated protein 1A/1B-light chain 3 (LC3).

Further agents of the invention, include, for example, one or more of anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). In some embodiments, a compound of Formulae I-IV is used adjunctively or neoadjunctively with these agents in the treatment of prevention of dry AMD and/or RPD.

Yet further agents of the invention are described, for example, in WO/2013/163758 and PCT/IB2015/053609, the entire contents of which are hereby incorporated by reference.

Still further agents of the invention, including in the context of dry AMD and RPD methods are found in Arq. Bras. Oftalmol. vol. 75 no. 1 January/February 2012, the entire contents of which are hereby incorporated by reference.

### Fluorescent Compounds

In some embodiments, the fluorescent compound is suitable for imaging with various wavelengths of fluorescence. In some embodiments, these wavelengths range from visible light to infrared, e.g., 390 nm to 1 mm, including, for example, blue light, white light, and near-infrared. In some embodiments, the dye is a near-infrared dye. In some embodiments, the dye is ICG.

In some embodiments, the fluorescent compound is suitable for imaging with various wavelengths of fluorescence. In some embodiments, these wavelengths range from visible light to infrared, e.g., about 390 nm to about 1 mm, including, for example, blue light, white light, and near-infrared. In some embodiments, the absorption is from about 390 nm to about 1 mm. In some embodiments, the emission is from about 390 nm to about 1 mm.

In some embodiments, the fluorescent compound absorbs light at a wavelength of about 600 nm to about 900 nm and/or emits light at a wavelength of about 750 nm to about 950 nm.

In some embodiments, the dye is a near-infrared dye. In some embodiments, the dye is ICG. In some embodiments, the ICG excitation/emission filters are 795 nm (excitation)/810 nm (emission).

In some embodiments, the dose of the fluorescent compound, e.g. a dye (including ICG), is an effective amount of the fluorescent compound. In various embodiments, the dose of ICG is from about 0.1 to about 10 mg/kg of an animal. In some embodiments, the dose is about 0.1, or about 0.3, or about 0.5, or about 1.0, or about 2.0, or about 3.0, or about 4.0, or about 5.0, or about 6.0, or about 7.0, or about 8.0, or about 9.0, or about 10.0 mg/kg of an animal.

In various embodiments, the fluorescent compounds, or metabolites thereof, cause a fluorescence which occurs in RPE cells and/or immune cells.

In other embodiments, the methods described herein do not comprise administering (i) an additional amount of fluorescent compound to the animal or (ii) a second fluorescent compound to the animal.

### Toxins

In some embodiments, a toxin known to affect ocular tissue, including but not limited to, the RPE or retina, is provided.

In some embodiments, the toxin is one or more of aluminum, aminophenoxyalkanes (a non-limiting example includes, but is not limited to, 1,4-bis(4-aminophenoxy)-2-phenylbenzene to rats), cationic amphophilic drugs/tricyclic antidepressants (non-limiting examples include but are not limited to amiodarone, chloroamitriptyline, chlorphentermine, clomipramine, imipramine, iprindole, various aminoglycosides, and other cationic amphophilic compounds), desferrioxamine, dl-(p-trifluoromethylphenyl) isopropylamine hydrochloride, fluoride (e.g. sodium fluoride), iodate (non-limiting examples include but are not limited to, sodium or potassium iodate), iodoacetate, lead, methanol and formic acid, 4,4′-Methylenedianiline, N-methyl-N-nitrosurea, naphthalene, napthol, nitroaniline (N-3-pyridylmethyl-N′-p-nitrophenylurea/nitroanilin/pyriminil), organophosphates (non-limiting examples include but are not limited to ethylthiometon, fenthion, and fenitrothion), oxalate (a non-limiting example includes but are not limited to dibutyl oxalate), phenothiazines (non-limiting examples include but are not limited to piperidylchlorophenothiazine, thioridazine, and chlorpromazine), quinolines (a non-limiting example includes, but is not limited to, chloroquine and hydroxychloroquine), streptozotocin, taurine deficiency, urethane, zinc deficiency caused by metal chelators, and derivatives and variants thereof.

In some embodiments, the toxin is an iodate. In some embodiments, the toxin is sodium or potassium iodate.

In some embodiments, the toxin induces atrophy of ocular tissue. In various embodiments, the atrophy comprises necrosis and/or apoptosis. In various embodiments, the atrophy comprising necrosis and/or apoptosis is of RPE cells. In some embodiments, the toxin reduces or modifies autophagy. In some embodiments, the toxin induces geographic atrophy and/or the expansion of geographic atrophy (GA). In some embodiments, the toxin induces one or more of the above-mentioned effects.

In some embodiments, the toxin is administered one time, or two times, or three times. In some embodiments, the toxin is administered one time, or two times, or three times, or four times, or five times. In some embodiments, a second, or third, or fourth, or fifth administration is within about a day, or 1 week, or 1 month of the first.

In some embodiments, the toxin administered may be may be one or more of the agents described herein. In various embodiments, the second, or third, or fourth, or fifth pulse is a modulator of autophagy, cell survival, cell death, proliferation, regeneration, and the like, as described herein and as known in the art.

In another embodiment, the methods described herein comprise administering (i) an additional amount of a first toxin to the animal and/or (ii) a second toxin to the animal. In some embodiments, the methods described herein comprise further comprise observing a reduction in the rate of formation, growth or expansion of patches of ocular tissue atrophy or patches of tissue loss.

In various embodiments, doses of the toxins are known to those in the art. For example, a suitable dosage may be in a range of about 0.1 mg/kg to about 100 mg/kg of body weight of the subject, for example, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg body weight, inclusive of all values and ranges therebetween.

In one embodiment, the toxin is NaIO₃ and the dose is about 50 mg/kg of body weight. In one embodiment, the toxin is NaIO₃ and the dose is about 45 mg/kg of body weight. In one embodiment, the toxin is NaIO₃ and the dose is about 30 mg/kg of body weight.

### Pharmaceutically Acceptable Salts and Excipients

Any agent described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compounds of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, an agent of the invention is in the form or a pharmaceutically acceptable salt. In some embodiments, the pharmaceutically acceptable salt is a sodium salt. In some embodiments, the compounds of Formulae I-IV are in the form of a pharmaceutically acceptable salt.

Further, any agent described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

### Formulations, Administration, and Dosing

Any agent described herein can take the form of solutions, suspensions, emulsion, intra-ocular injection, intra-vitreal injection, topical ophthalmic drops, sub-conjunctival injection, sub-Tenon's injection, trans-scleral formulations, tablets, pills, pellets, capsules, capsules containing liquids, powders, sustained-release formulations, suppositories, emulsions, aerosols, sprays, suspensions, or any other form suitable for use. In one embodiment, the composition is suitable for an intra-vitreal injection (see, e.g., ILUVIEN or similar forms) or implantation (see, e.g., RETISERT or similar forms). In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment, any agent described herein is formulated for ophthalmic administration, including, for example, intravitreal or intraocular administration, topical, and/or intravenous administration. Typically, compositions for administration comprise sterile isotonic aqueous buffer. Where necessary, the compositions can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as lignocaine to lessen pain at the site of the injection.

In one embodiment, any agent described herein is formulated in accordance with routine procedures as a composition adapted for intra-ocular administration.

In one embodiment, any agent described herein is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any agent described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

The ingredients may be supplied either separately or mixed together in unit dosage form, for example, as a pre-mixed solution, dry lyophilized-powder, or water-free concentrate in a hermetically sealed container such as an ampule, pre-filled syringe, or sachette indicating the quantity of active agent. Where any agent described herein is to be administered by intra-vitreal or intra-ocular delivery, it can be dispensed, for example, with a pre-filled syringe or injector, or in an ampule for withdrawal into a suitable syringe. Where any agent described herein is to be administered by infusion, it can be dispensed, for example, with an infusion bottle containing sterile pharmaceutical grade water or saline. Where any agent described herein is to be administered by injection, an ampule of sterile water for injection or saline can be provided so that the ingredients can be mixed prior to administration.

Any agent described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, nanoparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

Compositions can be prepared according to conventional mixing, granulating, coating or polymerization methods, respectively, and the present compositions can comprise, in one embodiment, from about 0.1% to about 99%; and in another embodiment from about 1% to about 70% of any agent described herein by weight or volume.

In another embodiment, any agent described herein acts synergistically when co-administered with another agent and is administered at doses that are lower than the doses commonly employed when such agents are used as monotherapy.

For example, the dosage any agent described herein as well as the dosing schedule can depend on various parameters, including, but not limited to, the ocular disorder being treated, the subject's general health, and the administering physician's discretion. Any agent described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional therapeutic agent, to a subject in need thereof. In various embodiments any agent described herein is administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, an agent of the invention, including compounds of Formulae I-IV, or their pharmaceutically acceptable salts, and one or more additional therapeutic agents are administered within 3 hours. In another embodiment, an agent of the invention, including compounds of Formulae I-IV, or their pharmaceutically acceptable salts, and one or more additional therapeutic agents are administered at 1 minute to 24 hours apart.

The amount of any agent described herein that is admixed with the carrier materials to produce a single dosage can vary depending upon the subject being treated and the particular mode of administration. In vitro or in vivo assays can be employed to help identify optimal dosage ranges.

In general, the doses that are useful are known to those in the art. For example, doses may be determined with reference Physicians' Desk Reference, 66th Edition, PDR Network; 2012 Edition (Dec. 27, 2011), the contents of which are incorporated by reference in its entirety.

The dosage of any agent described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular ocular disorder being treated, the severity of the disorder, and the anatomical location of the disorder. Some variations in the dosage can be expected.

In some embodiments, the administering is effected orally or intra-vascularly, or intraocularly, or periocularly, or to the ocular surface

When ophthalmically administered to a human, for example, intravitreally, the dosage of an agent of the invention, including, for example, Formulae I-IV, or a pharmaceutically acceptable salt thereof and/or additional therapeutic agent is normally 0.003 mg to 5.0 mg per eye per administration, or 0.03 mg to 3.0 mg per eye per administration, or 0.1 mg to 1.0 mg per eye per administration. In one embodiment, the dosage is 0.03 mg, 0.3 mg, 1.5 mg or 3.0 mg per eye. In another embodiment, the dosage is 0.5 mg per eye. The dosage can range from 0.01 mL to 0.2 mL administered per eye, or 0.03 mL to 0.15 mL administered per eye, or 0.05 mL to 0.10 mL administered per eye. In one embodiment, the administration is 400 μg of compound, monthly for at least three months.

Generally, when orally administered to a mammal, the dosage of any agent described herein may be 0.001 mg/kg/day to 100 mg/kg/day, 0.01 mg/kg/day to 50 mg/kg/day, or 0.1 mg/kg/day to 10 mg/kg/day. When orally administered to a human, the dosage of any agent described herein is normally 0.001 mg to 1000 mg per day, 1 mg to 600 mg per day, or 5 mg to 30 mg per day. In one embodiment, oral dosage is 600 mg per day. In one embodiment, the oral dosage is two 300 mg doses per day. In another embodiment, oral dosage is 7.5 mg per week to 15 mg per week.

For administration of any agent described herein by parenteral injection, the dosage is normally 0.1 mg to 250 mg per day, 1 mg to 20 mg per day, or 3 mg to 5 mg per day. Injections may be given up to four times daily. Generally, when orally or parenterally administered, the dosage of any agent described herein is normally 0.1 mg to 1500 mg per day, or 0.5 mg to 10 mg per day, or 0.5 mg to 5 mg per day. A dosage of up to 3000 mg per day can be administered.

In some embodiments, it may be desirable to administer one or more any agent described herein to the eye. Administration may be, by way of non-limiting example, intra-ocular, intra-vitreal, topical (including, but not limited to, drops and ointment), sub-conjunctival, sub-Tenon's, trans-scleral, suprachoroidal, subretinal, and via iontophoresis.

Other routes of administration may also be used, such as, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

The mode of administration can be left to the discretion of the practitioner, and depends in-part upon the site of the medical condition. In most instances, administration results in the release of any agent described herein into the bloodstream.

Any agent described herein can be administered orally. Such agents can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

Further methods of administration include but are not limited to intra-ocular, intra-vitreal, topical ocular (including but not limited to drops, ointments and inserts), sub-conjunctival, sub-Tenon's, suprachoroidal, trans-scleral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, more than one of any agent described herein is administered to the eye. Administration may be, by way of non-limiting example, intra-ocular, intra-vitreal, topical (including, but not limited to, drops and ointment), sub-conjunctival, sub-Tenon's, trans-scleral, and iontophoresis. The mode of administration can be left to the discretion of the practitioner, and depends in-part upon the site of the medical condition. In most instances, administration results in the release into the bloodstream.

In specific embodiments, it may be desirable to administer locally to the area in need of treatment.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989). In yet another embodiment, delivery can be in a controlled release system. In one embodiment, a slow release intra-ocular device may be used. In some embodiments, this device consists of a locally delivered erodible or non-erodable liquid, gel, polymer, etc.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In another embodiment, a controlled-release system can be placed in proximity of the target area to be treated, e.g., the retina, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any agent described herein can, independently, be one to four times daily or one to four times per month or one to six times per year or once every two, three, four or five years. Administration can be for the duration of one day or one month, two months, three months, six months, one year, two years, three years, and may even be for the life of the subject. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

The dosage regimen utilizing any agent described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any agent described herein can be administered in a single daily dose, or the total daily dosage can be administered in divided doses of two, three or four times daily. Furthermore, any agent described herein can be administered continuously rather than intermittently throughout the dosage regimen.

### Methods of Treatment

In various aspects, the present invention provides for a method for treating or preventing an ocular disorder described herein. In these aspects, the “agent of the invention” comprise compounds useful for both monotherapy and combination therapy (e.g. as an additional therapeutic agent).

In various aspects, the invention provides a method for treating or preventing an ocular disorder, comprising administering to a subject in need thereof an effective amount of an agent effective for the treatment of an ocular disorder in which such agent is an immunomodulatory agent (by way of non-limiting example, a MCP-1 and/or MCP-2 and/or MCP-3-modulating agent, including a compound of Formulae I-IV, a PPAR gamma modulator, a migration inhibitory factor (MIF) inhibitor, and a chemokine receptor 2 (CCR2) inhibitor. In some embodiments, the immunomodulatory agent targets macrophages. In some embodiments, the immunomodulatory agent modulates M1/M2 polarization. In some embodiments, the immunomodulatory agent modulates (e.g. reduces) M1 macrophage activity in the subject and/or modulates (e.g. increases or maintains) M2 macrophage activity in the subject. In some aspects, the invention relates to a method for treating or preventing an ocular disorder, comprising administering to a subject in need thereof an effective amount of an agent effective for the treatment of an ocular disorder, wherein the subject has an increased or decreased expression or activity of one or more of CD64, IDO, SOCS1, CXCL10 Marco, Socs3, Nos2, II12b, Ptgs2 (Cox2), II23a (II23p19), and Ido1.

In some embodiments, the subject has an increased or decreased expression or activity of one or more of MRC1, TGM2, CD23, CCL22 Relma (Fizz1, Retnla), Socs2, Irf4, Chia (Amcase), Chi3I1 (Gp39, Ykl40), Chi3I2 (Yk139), Chi3I3(Ym1), Cxcl13, Ccl12, Ccl24, and Klf4.

In some embodiments, the subject has an increased or decreased expression or activity of one or more of Ccl5, CD163, Cx3Cr1, FasIg, Gfap, Csf2, Icam1, Ifng, II10, II12b, II13, II17a, II18, II1b, II22, II4, II6, Klf4, Mrc1, Myd88, Nlrp3, Nos2, Ppary, Tgfb1, Tlr4, Tnf, Vcam1, Ccl2, Ccl5, Ccl7, Ccr2, Socs1, Socs3, Stat1, Stat3, and Stat6. In some embodiments, the subject has an increased or decreased expression or activity of one or more of Myd88, Klf4, Nlrp3, Ccl2, Ccl5, Ccl7, Socs1, Socs3, Stat1, Stat3, and Stat6.

In some embodiments, a compound of Formulae I-IV leads to differential gene expression of any of the following, directly or indirectly Ccl5, CD163, Cx3Cr1, FasIg, Gfap, Csf2, Icam1, Ifng, II10, II12b, II13, II17a, II18, II1b, II22, II4, II6, Klf4, Mrc1, Myd88, Nlrp3, Nos2, Ppary, Tgfb1, Tlr4, Tnf, Vcam1, Ccl2, Ccl5, Ccl7, Ccr2, Socs1, Socs3, Stat1, Stat3, and Stat6, optionally in macrophages or RPE directly. In some embodiments, a compound of Formulae I-IV modulates NFκB.

In one aspect, the present invention provides a method for treating or preventing an ocular disorder, comprising administering to a subject in need thereof an effective amount of a MCP-1-modulating agent. In some embodiments, the MCP-1-modulating agent targets the distal and/or proximal regulatory region of the MCP gene promoter/enhancer region(s). In some embodiments, the MCP-1-modulating agent targets the proximal regulatory region of the MCP-1 promoter. In some embodiments, present invention provides a method for treating or preventing an ocular disorder, comprising administering to a subject in need thereof an effective amount of a MCP-2 and/or MCP-3-modulating agent. In some embodiments, the MCP-2 and/or MCP-3-modulating agents optionally target the distal and/or proximal regulatory region of the MCP-2 and/or MCP-3 gene promoter/enhancer region(s). In some embodiments, the MCP-2 and/or MCP-3-modulating agents optionally target the proximal regulatory region of the MCP-2 and/or MCP-3 promoter. In some embodiments, the MCP-1 and/or MCP-2 and/or MCP-3-modulating agent is a compound of Formulae I-IV.

In one aspect, the present invention provides a method for treating or preventing an ocular disorder, comprising administering to a subject in need thereof an effective amount of a MCP-1-modulating agent. In some embodiments, the MCP-1-modulating agent targets the proximal regulatory region of the MCP-1 promoter. In some embodiments, present invention provides a method for treating or preventing an ocular disorder, comprising administering to a subject in need thereof an effective amount of a MCP-2 and/or MCP-3-modulating agent, MCP-2 and/or MCP-3-modulating agents optionally targeting the proximal regulatory region of the MCP-2 and/or MCP-3 promoter.

In some embodiments, the subject is not undergoing treatment with and/or is unresponsive to one or more of an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). In some embodiments, the subject has evidence of AMD as confirmed by the presence of at least 1 druse greater than about 125 μm in diameter. In some embodiments, the subject has no evidence of prior or active choroidal neovascularization (CNV). In some embodiments, the subject has one or more well-demarcated GA lesions of a total area of about 2 to about 20 mm² in one or more eye. In some embodiments, the subject has a best-corrected visual acuity score of greater than about 35 letters or a Snellen VA equivalent of about 20/200 or better. In some embodiments, the subject has early stage dry macular degeneration as evidenced by several small drusen or a few medium-sized drusen. In some embodiments, the subject has early stage dry macular degeneration as evidenced by a large number of medium-sized drusen or one or more large drusen. In some embodiments, the subject has early stage dry macular degeneration as evidenced by several large drusen and/or an extensive breakdown of cells in the macula.

An evaluation of any of the treatments disclosed herein can comprise optical imaging, including, by way of non-limiting example, cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared).

In some embodiments, the methods described herein comprise reducing the amount of pseudodrusen in the subject and/or reducing the amount of pseudodrusen in any one of the foveal area, perifoveal area, juxtafoveal area, and extrafoveal area of the subject's eye. In other embodiments, the methods described herein comprise reducing the rates of progression to late disease, wherein the late disease is any one of choroidal neovascularization or geographic atrophy. In some embodiments, the methods described herein comprise reducing the rates of expansion of geographic atrophy.

In some embodiments, the methods of treatment described herein comprise treatment, prevention, or reduction in the rate of pathogenesis of dry AMD and/or RPD.

### Compound Evaluation Methods

In some aspects, the invention provides a method for identifying whether a candidate compound is useful for the treatment of an ocular disorder, comprising (a) administering an effective amount of a test compound to an animal whose eye comprises (i) a fluorescent compound in an amount effective to indicate the presence of an ocular disorder in the animal and (ii) a toxin in an amount effective to induce atrophy of ocular tissue; (b) exposing the eye to light having a wavelength and intensity effective to cause the fluorescent compound to fluoresce; (c) comparing the eye's fluorescence pattern to a fluorescence pattern of an animal's eye that comprises the fluorescent compound and the toxin but not the test compound; and (d) selecting the test compound as a candidate compound if the result of the comparison of step (c) indicates that the test compound is useful for the treatment of an ocular disorder. In some embodiments, step (b) comprises exposing the eye to light having a wavelength and intensity effective to cause the fluorescent compound to fluoresce, whether performed coincidentally with administration of the fluorescent compound, or later administration of the fluorescent compound. In one aspect, the present invention provides a method of making an agent effective for the treatment of an ocular disorder, comprising (a) identifying the agent by (i) administering an effective amount of a test compound to an animal whose eye comprises (1) a fluorescent compound in an amount effective to indicate the presence of an ocular disorder in the animal and (2) a toxin in an amount effective to induce atrophy of ocular tissue; (ii) exposing the eye to light having a wavelength and intensity effective to cause the fluorescent compound to fluoresce; (iii) comparing the eye's fluorescence pattern to a fluorescence pattern of an animal's eye that comprises the fluorescent compound and the toxin, but not the test compound; and (iv) selecting the test compound as a candidate compound if the result of the comparison of step (iii) indicates that the test compound is useful for the treatment of an ocular disorder and (b) formulating the identified agent for administration to the eye. In some aspects, there is provided a method for treating or preventing an ocular disorder, comprising administering to a subject in need thereof a pharmaceutical composition comprising an effective amount of an agent of any one of the agent effective for the treatment of an ocular disorder as made in using the method described above and a pharmaceutically acceptable carrier or excipient. Illustrative agents effective for the treatment of an ocular disorder are enumerated herein (e.g. as “agents of the invention”).

In other embodiments, the comparing occurs at least about 24 hours, or at least about 7 days, or at least about 30 days, or at least 60 days, or at least 90 days after administering the test compound. In other embodiments, the comparing occurs at least about 2 months, or about 3 months, or about 4 months, or about 5 months, or at a maximum about 6 months. In some embodiments, the comparing comprises observation of the eye of the same animal pre- and post-administering an effective amount of a test compound. In some embodiments, the comparing comprises observation of the eye of different animals under different conditions (e.g. with or without administering an effective amount of a test compound).

In still other embodiments, the methods further comprise the step of observing the eye prior to administering the test compound. In some embodiments, this observing establishes one or more pre-administration characteristics of the eye.

In some embodiments, the comparison and/or observation comprises evaluating optical imaging, including, by way of non-limiting example, cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared), between two different conditions (e.g. with or without administering an effective amount of a test compound).

In some embodiments, a compound is useful for the treatment of an ocular disorder if it provides treatment, prevention, or reduction in the rate of pathogenesis of an ocular disorder.

In yet another embodiment, the methods described herein comprise administering the fluorescent compound prior to administering the test compound. In still another embodiment, the methods described herein do not comprise administering (i) an additional amount of fluorescent compound to the animal or (ii) a second fluorescent compound to the animal.

In other embodiments, the methods described herein comprise administering the toxin prior to administering the test compound and/or administering the toxin prior to administering the fluorescent compound.

In other embodiments, a plurality of candidate compounds is identified. In some embodiments, the methods described herein further comprise comparing the usefulness of the plurality of candidate compounds in the treatment of an ocular disorder and selecting a lead compound based on the comparison. In some embodiments, a lead compound is a preferred compound among a plurality of candidate compounds.

In some embodiments, the comparison comprises evaluating optical imaging, including, by way of non-limiting example, cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared), between two different conditions (e.g. with a first candidate compound versus with a second candidate compound). More than two candidate compounds can be compared.

### Diagnostic and Predictive Methods

In some aspects, the invention provides a method for identifying a subject who has an ocular disorder and is more likely than not to respond to treatment with an agent comprising determining whether the subject's eye has, or previously had, an increase (including a transient increase) in permeability across the epithelial barrier between a choroid and a retina of the eye relative to an undiseased state; wherein the increase in permeability indicates that the subject is more likely than not to respond to treatment with the agent; and wherein the agent is selected from a compound of Formulae I-IV (as described herein) or a pharmaceutically acceptable salt thereof.

In another aspect, the present invention provides a method for identifying an ocular disorder subject who is more likely than not to respond to treatment with an agent comprising determining whether the subject's eye has an presence (e.g. an influx) of phagocytic immune cells across a RPE (and/or from the inner retina), relative to an undiseased state, wherein the presence of phagocytic immune cells indicates that the subject is more likely than not to respond to treatment with the agent; and wherein the agent is selected a compound of Formulae I-IV (as described herein) or a pharmaceutically acceptable salt thereof.

In another aspect, the invention provides a method for determining whether an ocular disorder in a subject is responsive to treatment with an agent that inhibits the function of a subject's immune cells, comprising detecting a presence, detecting an absence, or measuring an amount of immune cells in the subject's eye, wherein the subject's eye fluoresces in response to light having a wavelength of about 600 nm to about 900 nm, or about 400 nm to about 900 nm, or about 400 to about 1600 nm.

In some embodiments, the methods described herein further comprise administering to the subject an effective amount of a fluorescent compound, wherein the detecting or measuring occurs at least one day after the administration of the fluorescent compound. In some embodiments, the detecting or measuring occurs at least one day after administering to the subject an effective amount of a fluorescent compound.

In some embodiments, the methods described herein comprise DNIRA for determining whether an ocular disorder in a subject is responsive to treatment with an agent that inhibits the function of a subject's immune cells.

In some embodiments, the methods described herein further comprise the step of detecting or measuring FAF in the eye of the subject. In some embodiments, the methods described herein further comprise the step of correlating an FAF pattern to the presence, absence, or amount of immune cells in the subject's eye. In some embodiments, the methods described herein comprise a correlating between FAF and DNIRA data. In some embodiments, the correlating is of the spatial patterns observed in FAF and the subject's eye fluorescence in response to light having a wavelength of about 600 nm to about 900 nm, or about 400 nm to about 900 nm, or about 400 to about 1600 nm.

In some embodiments, areas of hyperfluorescent FAF or abnormal patterns of FAF may spatially coincide with areas of abnormal DNIRA, which may be hypofluorescent or hyperfluorescent. As abnormal FAF or hyperfluorescent FAF coincides with areas of disease activity and can predict areas of atrophy, without wishing to be bound by theory, in embodiments in which DNIRA labels the RPE and spatially coincides with hyperfluorescent FAF or abnormal FAF, phagocytic RPE cells that ingest s dye (for example, ICG) have abnormal amounts of lipofuscin or lipofuscin-like material. Further, without wishing to be bound by theory, in embodiments in which DNIRA labels immune cells (e.g. phagocytic immune cells, or cells of the innate immune system), and coincides spatially with hyperfluorescent FAF or abnormal FAF, phagocytic immune cells that ingest a dye (for example, ICG) have abnormal amounts of lipofuscin of lipofuscin-like material and/or coincide with cells that have abnormal amounts of lipofuscin or lipofuscin-like material. In some embodiments, the co-localization of abnormal FAF with abnormal DNIRA can therefore identify cellular targets for therapy. Such co-localization, provides, in some embodiments, both in animal models of disease, and in patients, the ability to target the immune system to reduce, slow, prevent disease progression.

In some embodiments, the detecting or measuring occurs at about one day, or about seven days, or at about thirty days after administration of the fluorescent compound. In other embodiments, the comparing occurs at least about 2 months, or about 3 months, or about 4 months, or about 5 months, or at a maximum about 6 months. In some embodiments, the comparing comprises observation of the eye of the same animal pre- and post-administering an effective amount of a test compound. In some embodiments, the comparing comprises observation of the eye of different animals under different conditions (e.g. with or without administering an effective amount of a test compound).

In some embodiments, the comparison comprises evaluating optical imaging, including, by way of non-limiting example, cSLO, FAF, OCT (including with cross-sectional, three-dimensional and en face viewing), SD-OCT (with cross-sectional, three-dimensional and en face viewing), or other imaging modalities including other wavelengths of fluorescence (e.g. wavelengths ranging from blue to infrared, e.g., 390 nm to 1 mm, including, for example, blue light, white light, red-free, near infra-red, or infrared), between two different conditions (e.g. with or without administering an effective amount of a test compound).

In some embodiments, the methods described herein do not further comprise administering (a) an additional amount of the fluorescent compound or (b) a second fluorescent compound.

Subjects and/or Animals

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell, such as, for example, an RPE cell and/or an immune cell. In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

In various embodiments, a subject's and/or an animal's eye comprises (i) a fluorescent compound in an amount effective to indicate the presence of an ocular disorder in the subject and/or animal and (ii) a toxin in an amount effective to induce atrophy of ocular tissue. In some embodiments, such a subject and/or animal is administered an agent of the invention or is not administered an agent of the invention.

In various embodiments, RPE and immune cells are evaluated and/or effected. In some embodiments, immune cells include cells of a subject's and/or animal's innate immune system. In some embodiments, such cells include, but are not limited to, macrophage, monocyte, and microglial cells. In various embodiments, the invention provides for detecting a presence, detecting an absence, or measuring an amount of immune cells in a subject's and/or animal's eye Kits

The invention provides kits that can simplify the administration of any agent described herein. An exemplary kit of the invention comprises any agent described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the ocular surface. The kit can also further comprise one or more additional agent described herein.

In one embodiment, the kit comprises a container containing an effective amount of an agent of the invention, including, for example, compound of Formulae I-IV, or a pharmaceutically acceptable salt thereof and an effective amount of another therapeutic agent, such those described herein.

The invention is further described by the following non-limiting examples.

### EXAMPLES

### Example 1: In Vivo Therapeutic Effects of the Compounds of Formulae I-IV

The in vivo therapeutic effects of the compounds of Formulae I-IV are tested. Specifically, a model of non-exudative “dry” AMD and/or Reticular Pseudodrusen (RPD) and/or late onset retinal degeneration (LORDs) is utilized to evaluate whether compounds of Formulae I-IV are able to protect the retina and retinal pigment epithelium (RPE) against toxin-induced damage. The ability of the compounds of Formulae I-IV to protect against the development of patches of RPE and photoreceptor loss known as Geographic Atrophy (GA) is also examined. Additionally, the activity of the compounds of Formulae I-IV in modulating the macrophage inflammatory response is assessed.

### Methodology:

Animal care: Animals are handled in accordance with the Association for Research in Vision and Ophthalmology (ARVO) guidelines for the humane use of animals in ophthalmic research, and according to the Canadian Council on Animal Care guidelines. For all experiments, SD rats aged 6-10 weeks are kept at a 12 hour dark/light cycle, with food and water ad libitum. For evaluations, animals are anesthetized with a combination of ketamine (100 mg/kg) and xylazine (10 mg/kg), and pupils dilated with a single drop of 0.8% tropicamide in 5% phenylephrine hydrochloride solution (Diophenyl-T, Sandoz Canada Inc). GenTeal lubricating eye drops (Novartis, Canada) are repeatedly applied to the corneal surface during all procedures.

In vivo imaging: In vivo images are acquired using a commercially available confocal scanning laser ophthalmoscope (cSLO) (Heidelberg Retinal Angiography, HRA-2, Heidelberg Engineering, Germany). Images are obtained in the red-free, FAF (488/500 nm excitation/emission), IR reflectance channel (830 nm) and ICG fluorescence channels (795/810 nm excitation/emission). Animals are pre-screened using cSLO, and those with spontaneous defects are eliminated from the study. The surface of the cornea is kept moist with repeated application of Genteal lubricating drops (Alcon, Mississauga, Canada) during all in vivo imaging. Multiple images are taken of each eye starting at the optic nerve head (ONH), and moving in concentric rings to the mid- and far-periphery where possible. In some cases, due to difficulties in aligning the cSLO with the small pupil, contiguous images are obtained in a hemi-retina only, with re-alignment of the animal for subsequent imaging of the second half. Composite images of the fundus are assembled post-hoc in PowerPoint and exported for analysis.

Angiography: ICG dye (Cardiogreen, Sigma) is freshly prepared prior to experimentation to a final stock concentration of 5.0 mg/ml in sterile water. A 24-gauge catheter is inserted into the tail vein, and ICG dye is infused at doses of 0.35, or 5.0 mg/kg. Images are taken prior to injection (baseline), during dye circulation, and at various intervals thereafter out to 20 minutes.

Delayed Near Infra Red Analysis (DNIRA): DNIRA images are obtained in the days and weeks after ICG injection using the ICG angiography settings, with excitation/emission filters in place, but without re-injecting the dye after day 0. Images are taken 2-120 days after angiography.

Quantification of FAF images: Composite images are grouped and imported to Image J (National Institutes of Health) for analysis by a masked observer. Comparison between the size of the patch of damage, determined from different images at different dosages of toxin, is achieved by normalizing the image size using the central (optic nerve head) cSLO image, as reference. An assumption is made that the magnification between fundus images does not vary significantly between rats. Areas with abnormal FAF signal are manually outlined by a masked reader using the “polygon” tracing tool, and the regions of interest are measured in pixels. For dose-response analysis, the size of the patches is compared against the average of patches from 45 mg/kg NaIO₃ (assigned a value of 100 units).

Electroretinography (ERG): The bright-flash ERG response is evaluated in animals using the Espion (DiagnosysLLC, USA) mini-Ganzfeld system following high (5.0 mg/kg) and low (0.35 mg/kg) dose administration of ICG, or saline control. Following anesthesia, animals are placed on an electrically silent heating pad and gold coil electrodes placed at the edge of the cornea after application of GenTeal lubricating drops. Following a short train of dim flashes (0.01 candela s/m2, 1.0 Hz), the photopic b-wave response is evaluated using a single bright flash (3 candela s/m2) that is previously determined to consistently approximate the maximum b-wave amplitude.

Sodium iodate: The RPE toxin sodium iodate (NaIO₃, Sigma) is prepared fresh weekly at a stock solution of 45 mg/ml in saline (Baxter, Mississauga, Canada). This solution is diluted to final concentrations of 5 mg/ml, 15 mg/ml, 30 mg/ml and 45 mg/ml, such that all animals receive the same volume to achieve final dosages of 5 mg/kg to 45 mg/kg. NaIO₃ is injected systemically via a 24-gauge catheter inserted into the tail vein. ICG injection (0.35 mg/kg) and angiography are performed at day 0 immediately prior to NaIO₃ injection. To test the effects of compounds of Formulae I-IV, a standardized dose is used.

Sodium iodate and nitroglycerin infusions: Sodium iodate (Sigma) is prepared fresh at a concentration of 45 mg/ml in 0.9% saline for each set of experiments, and injected to a final concentration of 45 mg/kg body weight using a 24 gauge catheter inserted into the tail vein. In a subset of animals, nitroglycerin (clinical grade, St. Michael's Hospital) is infused at 2 μg/kg/min for over 30 minutes using a tail vein catheter and an animal infusion pump system, and a second dose of NaIO₃ is injected subsequent to infusion.

Intra-ocular injection of compounds of Formulae I-IV: intra-vitreal injection of compounds of Formulae I-IV is performed under a dissecting microscope (SMZ800; Nikon Instruments, Melville, USA) with a Hamilton syringe (Cat#7634-01, Hamilton Company, Reno, USA). A small incision is made behind the limbus using a beveled 33-gauge needle (BD, Mississauga, Canada). A 30-gauge blunt needle is then inserted and at an angle of approximately 45° to the pupillary axis. Two (2) μL of a solution including compounds of Formulae I-IV or control solution (saline) is manually injected.

Autofluorescent fluorescence microscopy: Freshly excised wholemount ocular sample, either retinal or posterior cup devoid of retina, are evaluated using an epifluorescent microscope with wavelength of 488 nm/512 nm.

Immunofluorescence microscopy: Immunohistochemistry is performed using standard protocols. Briefly, cells are fixed with 4% PFA/PBS for 15 minutes, and blocked with 1.25% BSA in TBS for 30 minutes at room temperature. Primary rabbit anti-ZO-1 is used at 2.5 μg/ml in 1.25% BSA/TBS and incubated for 1 hour at room temperature. Cells are washed in TBS and incubated with goat anti-rabbit 488 (Invitrogen) at 5 μg/ml for 1 hour at room temperature, washed, and counterstained with To-Pro-3 (Invitrogen). Some RPE monolayers are stained with 647 nm conjugated phalloidin (Invitrogen) for 20 minutes at room temperature and counterstained with Sytox green nuclear stain (Invitrogen). Slides are mounted with fluorescent mounting medium (Dako) and images acquired using a Leica TCS SL confocal fluorescent microscope (Leica Microsystems, GmbH, Wetzlar, Germany), with Leica Confocal Software V 2.61.

RNA isolation, Qiagen RT2 Profiler PCR array, and data analysis: Total RNA is isolated using 8 eyes at evolution stage following NaIO3 injection either with treatment with a compound of Formulae I-IV, or saline vehicle control, from rat retinas and posterior eyecups using Trizol Reagent (Life Technologies, USA). Briefly, after enucleation of rat eye balls, they are dissected in cold RNAlater Solution (Life Technologies, USA). The entire retina is then removed and stored in RNAlater solution, and RPE and choroid layers are scraped with a surgical scalpel blade and combined with the retina. Manufacturer's protocol is followed for RNA extraction with the Trizol method. Total RNA is further purified with RNeasy MinElute Cleanup kit (Qiagen, USA). Their quantity and quality are checked with a Nanodrop 2000 spectrophotometer (Thermo Scientific, USA) and Agilent 2100 Bioanalyzer (Agilent Technologies, USA).

cDNA is synthesized using RT2 First Strand Kit (Qiagen, USA) with 2 μg of total RNA per reaction. Real-time PCR is performed using Qiagen 384-well Custom RT2 Profiler PCR Array and RT2 SYBR Green ROX qPCR Mastermix (Qiagen, USA) with 10 ng cDNA per well. The reactions are run on a ViiA 7 PCR system (Life Technologies, USA) with default cycling conditions. Differences in gene expression are analyzed using the Qiagen RT2 Profiler PCR Array Data Analysis Software V3.5 Web Portal (http://pcrdataanalysis.sabiosciences.com/pcr/arrayanalysis.php) based on AACT method and normalized to Peptidyl-prolyl cis-trans isomerase H (PPIH) as a reference gene. Results are reported as fold-change (2-AACT) normalized to baseline as 1-fold. P values are calculated based on a Student's t-test of the replicate 2-AACT values for each gene in the control group and treatment groups, and P values less than 0.05 are considered significant.

The primer sequences are proprietary. The amplicon size, Qiagen gene catalog number and primer reference position found below.

Qiagen RT² PCR array gene information.

| | | Reference position | Ampli- |
|---|---|---|---|
| | | of amplicon start in | con |
| | | gene sequence rela- | Size |
| Gene | Qiagen Gene Catalog Number | tive to gene start | (bp) |
| Ccl2 | PPR06714B (NM_031530) | 119 | 135 |
| Ccr2 | PPR06437A (NM_021866) | 477 | 160 |
| Ifng | PPR45050C (NM_138880) | 275 | 71 |
| II12b | PPR06446A (NM_022611) | 962 | 111 |
| II1b | PPR06480B (NM_031512) | 820 | 77 |
| Nlrp3 | PPR56639A (NM_001191642) | 3297 | 142 |
| Nos2 | PPR44835A (NM_012611) | 3382 | 165 |
| Socs3 | PPR06602A (NM_053565) | 144 | 145 |
| Stat1 | PPR48601A (NM_032612) | 2198 | 84 |
| Tnf | PPR06411F (NM_012675) | 262 | 86 |
| CD163 | PPR44023A (NM_001107887) | 3255 | 98 |
| II10 | PPR06479A (NM_012854) | 34 | 102 |
| II13 | PPR06485A (NM_053828) | 38 | 114 |
| II4 | PPR56680A (NM_201270) | 453 | 90 |
| Klf4 | PPR43919A (NM_053713) | 1383 | 148 |
| Mrc1 | PPR44294A (NM_001106123) | 4313 | 92 |
| Pparg | PPR47599A (NM_013124) | 1389 | 190 |
| Socs1 | PPR52395A (NM_145879) | 25 | 191 |
| Stat3 | PPR44745C (NM_012747) | 2198 | 122 |
| Stat6 | PPR43717A (NM_001044250) | 2466 | 191 |
| Tgfb1 | PPR06430B (NM_021578) | 1267 | 122 |
| Ccl7 | PPR06685B (NM_001007612) | 187 | 97 |
| Cx3cr1 | PPR06709A (NM_133534) | 959 | 122 |
| Gfap | PPR52927G (NM_017009) | 845 | 67 |
| Csf2 | PPR49732A (NM_053852) | 300 | 99 |
| II17a | PPR57758A (NM_001106897) | 470 | 190 |
| II18 | PPR06394A (NM_019165) | 496 | 113 |
| II22 | PPR63374A (NM_001191988) | 159 | 103 |
| Myd88 | PPR48967A (NM_198130) | 838 | 133 |
| Rpe65 | PPR52080B (NM_053562) | 659 | 125 |
| Ppih | PPR57387B (XM_001073803) | 127 | 54 |

Patients with AMD start “dry” and have small deposits in the macula known as drusen. Late in disease progression, patients convert to advanced disease which is characterized by the patchy loss of the outer retina and RPE. It is expected that treatment with compounds of Formulae I-IV provides secondary prevention against the late blinding complications of dry AMD and RPD known as Geographic Atrophy (GA). The purpose of secondary prevention is to maintain central visual acuity and central visual field prior to loss.

Briefly, a rodent model for dry AMD is used in which animals are challenged with a toxin to induce formation of, and prompt expansion of, a patch of geographic atrophy (GA). Geographic atrophy, a breakdown in the retinal pigment epithelium (RPE) and subsequent overlying retinal tissue, is a hallmark of dry AMD. Its expansion is an FDA-approved clinical endpoint. In this model, toxin is applied to establish patches of GA which can then be made to expand and therefore mimic the clinical progression of dry AMD. In this model, prevention can be studied by administering the compounds of Formulae I-IV prior to NaIO₃ toxic insult. The effectiveness of treating GA can be studied by administering the compounds following NaIO₃ insult.

Using this unique animal model, it is expected that administration of the compounds of Formulae I-IV prevents and protects against NaIO₃-induced retinal damage in vivo in a dose dependent manner. Histology shows that administration of the compounds preserved RPE cells and RPE layer integrity. RPE protection may be indicated by higher RPE65 gene expression. It is also expected that the compounds reduce the growth (expansion) of patches of GA, protect against patch development, and prevent disruption of the outer retina thereby preserving retinal function. Accordingly, the compounds of Formulae I-IV can prevent both the initiation and the expansion of retinal damage and serves to both prevent and protect against GA.

To validate the efficacy of the compounds of Formulae I-IV to treat dry AMD, another animal model is used in which a secondary toxic insult is used to damage the retina even further in addition to the NaIO3-induced damage so as to speed up the expansion of atrophy. Specifically, nitroglycerin (NTG) is used and is infused at the evolution stage of atrophy alongside a second dose of NaIO₃ to result in an expanded patch of atrophy into the region where the retina is previously healthy. It is expected that administration of a compound of Formulae I-IV alongside the secondary toxic insult will prevent further expansion of atrophy in this model.

Compounds of Formulae I-IV may also act to reduce inflammatory activity by, for example, reducing “inflammation-associated” transcription. Conventionally, macrophages are capable of polarizing towards a classically activated M1 response or an alternatively activated M2 response. In a simplified view, M2 cells provide housekeeping functions such as scavenging debris and are suggestively good, while M1 cells are aggressive and propagate inflammation and are suggestively bad. It is expected that compounds of Formulae I-IV may reduce Monocyte Chemoattractant protein (MCP)-1 expression thus preventing upregulation of pro-inflammatory genes (including M1) genes following an inflammatory stimulus. Exemplary M1 genes that may be reduced following administration of compounds of Formulae I-IV include, for example, Ccl7 (MCP-3), SOCS3, and STAT1. Accordingly, the compounds may function to reduce the M1 macrophage response by preventing upregulation of the M1 gene expression, thereby tipping the balance in favor of M2. Additionally, compounds of Formulae I-IV can reduce the number of inflammatory cells that accumulate in any damaged areas.

### Example 2: RPE Protection and Complement Modulation

This example investigates if compounds of Formulae I-IV can directly influence MCP-1 expression in RPE cells independent of their activity on macrophages. MCP-1 is reported to be expressed by RPE, and is a potent Danger Associate Molecular Protein (DAMP) that activates M1 macrophages.

Specifically, an immortalized, telomerase negative human RPE cell line (hTERT-RPE1) is used to test the activities of the compounds of Formulae I-IV. Results are expected to show that compounds of Formulae I-IV can directly influence the hTERT-RPE1 cells by downregulating MCP-1 mRNA and the M1 transcriptome as well as potentially downregulating the KFκB protein. Compounds of Formulae I-IV may also protect hTERT-RPE cell viability when these cells are challenged with NaIO₃. Without wishing to be bound by theory, it is believed that compounds of Formulae I-IV have the potential to modulate the innate immune response both directly through their influence on macrophage polarization and indirectly by reducing the level of RPE signaling that initiates local response.

Age related macular degeneration is associated with deficiencies in innate immunity, and promising therapies target these pathways. The polymorphism that confers greatest risk for the development of AMD is complement factor H (CFH), a component of the alternate complement cascade. Cfh is a negative-regulator of complement and its absence leads to an accelerated response. Downstream members include C5 and C3, both of which have been targeted unsuccessfully in clinical trials. However, a recent promising phase II study using the complement factor D (CFD) inhibitor, Lampilizumab, suggests that CFD inhibition can slow the growth, i.e. expansion, of geographic atrophy over an 18 month period. This effect was most robust in patients with concurrent complement factors H and I polymorphisms. Normalized against baseline (pre-toxin), mRNA levels of many complement genes are known to increase or decrease by more than 2 or 3 fold following NaIO₃ administration. Major changes include C1, C3, C5 and C9, and of note, CFH and CFI both decrease. This latter finding may correlate with the finding that the CFD inhibitor, Lampilizumab, was most efficacious in patients with polymorphisms in these genes.

Compounds of Formulae I-IV are expected to influence complement gene expression including C1qb, C1qtnf5, C2, C3, C4b, C5, C6, C7, C8a, C9, Cfb, Cfd, Cfh, and Cfi. Accordingly, compounds of Formulae l-IV can exert their therapeutic effects by altering mRNA response and working synergistically with modulators of the complement system.

### Example 3: Treatment of Diabetic Retinopathy

Inflammation is suggested to play a central role in diabetic eye disease, including both diabetic retinopathy and diabetic macular edema. Cytokines, chemokines and other pro-inflammatory or pro-permeability molecules are described in human ocular tissues such as vitreous or aqueous, and also in serum. Animal models confirm an inflammatory response in pre-clinical studies.

Amongst the 50+ chemokines identified to date, it is reported that approximately 50% are altered in diabetes. Amongst these are MCP-1/ccl2 and RANTES. Transcription factors such as NFKb are also implicated in diabetic eye disease, and can be directly activated in the face of obesity and excess free fatty acids. Increased IKKb phosphorylation leads to NFkb translocation to the nucleus and activation of the inflammatory transcriptome. Further, serum levels of MCP-1/ccl2 and CRP are elevated in patients with severe diabetic retinopathy, potentially arising from systemic release of eye-derived mediators or, concurrent advanced systemic disease. Agents that can reduce these systemic factors, either locally or systemically might therefore be efficacious in the treatment of diabetic eye disease.

The effects of the compounds of Formulae I-IV on inflammatory mediators involved in diabetic eye disease are tested. Specifically, sodium iodate, at a high dose of 45 mg/kg was used to induce an inflammatory response that was treated with either high or low dose of compounds of Formulae I-IV administered intra-vitreally. The effects of the compounds are assessed at day 16 after toxin injection. It is expected that compounds of Formulae I-IV will reduce the various inflammatory mediators involved with diabetic eye disease. It is also expected that compounds of Formulae I-IV may reduce NFkb translocation to the nucleus.

In addition, the effects of hyperglycemia on isolated human RPE cells are addressed. In the presence of hyperglycemia (i.e., elevated serum glucose) and in the absence of insulin, most cells become hypoglycemic. This is also true for cells that are non-responsive or resistant to insulin. However, cells of the brain and retina uptake high levels of glucose in the absence of insulin and so become hyperglycemic. At low levels this can stimulate cell activity, however at higher levels glucose become toxic, inciting inflammation and leading to the formation of advanced glycated endproducts (AGEs). The immortalized human cell line, hTERT, is incubated with normal, medium-high and very high concentrations of D-glucose. Cell activity, measured in fluorescence unit is normalized against standard concentrations of glucose; L-glucose served as osmotic control. Cells are incubated for five days, with or without compounds of Formulae I-IV. It is expected that administration of the compounds of Formulae I-IV normalizes cellular stress in hTERT cells incubated with high glucose suggesting that these compounds are efficacious for the treatment of diabetic retinopathy.

### Example 4: Treatment of Dry AMD and/or RPD with Compounds of Formulae I-IV

Human subjects, 56 to 100 years of age or more, present with dry AMD, as diagnosed by one or more of the following clinical tests: clinical examination, FAF (at any wavelength), near infrared and/or red-free photography, fluorescein angiography, which allows for the identification and localization of abnormal vascular processes; OCT, which provides high-resolution, cross-sectional or en face images from within optical scattering media, such as the human retina and choroid; and structured illumination light microscopy, using a specially designed high resolution microscope setup to resolve the fluorescent distribution of small autofluorescent structures (lipofuscin granulae) in retinal pigment epithelium tissue sections.

The subjects are administered a compound of Formulae I-IV, in oral doses (e.g. 300 mg) once a day for 12 weeks. After an initial twelve-week treatment period, the subjects are evaluated for clinical outcomes. Alternatively, patients receive intravitreal injection of a vehicle containing a compound of Formulae I-IV, with or without a drug delivery vehicle.

A first clinical outcome is determined using a standard visual acuity test, as is well known in the art. The subjects are assessed for the ability to clearly see symbols and objects on a Snellen eye chart from a distance.

A second clinical outcome assesses the rate of progression of geographic atrophy. To do so, the subjects' pupils are dilated with 1.0% tropicamide and 2.5% phenylephrine before retinal imaging. Imaging is carried out with an instrument (e.g., Spectralis HRA+OCT; Heidelberg Engineering, Heidelberg, Germany) that allows for simultaneous recording of cSLO and spectral-domain optical coherence tomography (SD-OCT) with two independent scanning mirrors, as described in Helb, et al. Acta Ophthalmol. 2010 December; 88(8):842-9. Five modes of operation are employed: white light, red-free light, near infrared, FAF and OCT.

cSLO images are obtained according to a standardized operation protocol that includes the acquisition of near-infrared reflectance (A=815 nm) and FAF (excitation at A=488 nm, emission 500-700 nm) images. Simultaneous SD-OCT imaging is carried out with an illumination wavelength of 870 nm, an acquisition speed of 40,000 A-scans, and a scan depth of 1.8 mm. Two SD-OCT scans, one vertical and one horizontal, per eye are performed through the approximate foveal center, or in the case of RPD, in proximity to the vascular arcades of the macula. Fluorescein angiography (A=488 nm, emission 500-700 nm, 10% fluorescein dye) is performed as needed. Color fundus photographs are obtained with a fundus camera (e.g. FF 450 Visupac ZK5; Carl Zeiss Meditec AG, Jena, Germany).

Interpretation of clinical outcome data informs a decision for further treatment, if any.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed is:

1. A method for treating or preventing an ocular disorder, comprising administering to a subject in need thereof an effective amount of a compound of Formula I: 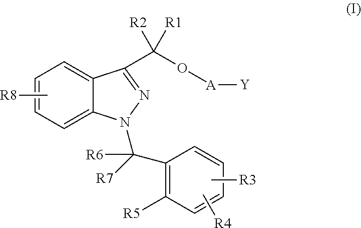
or a pharmaceutically acceptable salt thereof,
wherein
A may be a bond σ, —X₁— or —X₁—O—X₂—, in which
X₁ and X₂, which may be identical or different from each other, may be an alkyl group having from 1 to 5 carbon atoms, optionally substituted with one or more alkyl groups having from 1 to 5 carbon atoms or one or more alkoxy groups having from 1 to 3 carbon atoms,
Y is H when A is a bond σ, or Y may be H, —OH, or —N(R₁₁)(R₁₂), when A is —X₁— or —X₁—O—X₂—, in which
R₁₁ may be hydrogen, an alkyl group having from 1 to 5 carbon atoms, or R₁₁ together with R₁₂ forms a 4- to 7-membered heterocycle,
R₁₂ may be hydrogen, an alkyl group having from 1 to 5 carbon atoms, or R₁₂ together with R₁₁ forms a 4- to 7-membered heterocycle,
R₁ and R₂, which may be identical or different from each other, may be hydrogen, an alkyl group having from 1 to 5 carbon atoms,
R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,
R₅ may be hydrogen, an alkyl group having from 1 to 5 carbon atoms, an alkoxy group having from 1 to 3 carbon atoms, a halogen atom, —OH, —N(R)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms, and
R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms, and a pharmaceutically acceptable carrier or excipient.

2. The method of claim 1, wherein the compound is a compound of Formula I with the proviso that when A is a bond, and Y, R₁, R₂, R₆, and R₇ are hydrogen atoms,
if R₈ is a hydrogen atom, then the group linked to the nitrogen atom in the 1-position of the indazole ring is different from a benzyl group, a 4-chlorobenzyl group, or a 2-4-dichlorobenzyl group,
if R₈ is a fluorine atom in the 5-position of the indazole ring, then the group linked to the nitrogen atom in the 1-position of the indazole ring is different from 5-chloro-2-methoxybenzyl group, and
if R₈ is a trifluoromethyl group in the 6-position of the indazole ring, then the group linked to the nitrogen atom in the 1-position of the indazole ring is different from a 2-4-dichlorobenzyl group.

3. The method of claim 1 or 2, wherein the compound is an immunomodulatory agent.

4. The method of any one of the above claims, wherein the compound targets macrophages.

5. The method of any one of the above claims, wherein the compound modulates M1 macrophage activity in the subject.

6. The method of any one of the above claims, wherein the compound modulates M2 macrophage activity in the subject.

7. The method of any one of the above claims, wherein the compound is a MCP-modulating agent.

8. The method of any one of the above claims, wherein the compound differentially targets the proximal and/or distal regulatory region of the MCP-1 gene.

9. The method of any one of the above claims, wherein the compound is a small molecule with an indazole core.

10. The method of any one of the above claims, wherein the ocular disorder is one or more of dry age-related macular degeneration (AMD), reticular drusenoid disease (RPD), white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), late onset retinal degeneration (LORDs), and central serous retinopathy (CSR).

11. The method of any one of the above claims, wherein the ocular disorder is one or more of a diabetic eye disease (e.g. diabetic retinopathy and DME), Vogt-Kayanagi-Harada disease (VKH), Sarcoid uveitis, Ocular histoplasmosis, Presumed Ocular Histoplasmosis Syndrome, Autoimmune uveitis, Uveitis associated with systemic diseases (e.g. lupus), Crohn's disease, rheumatoid arthritis, and other diseases of known immune origin, Posterior uveitis, Anterior uveitis (e.g. iritis), Bechet's disease, Polyarteritis nodosa, and Wegener granulomatosis.

12. The method of any one of the above claims, wherein the subject has abnormal expression or activity of one or more of CD64, IDO, SOCS1, CXCL10 Marco, Nos2, II12b, Ptgs2 (Cox2), II23a (II23p19), Ido1, Adipoq, Ccl20, IL17 (subtype a) Ccl5, CD163, Cx3Cr1, FasIg, Gfap, Csf2, Icam1, Ifng, II10, II12b, II13, II17, II18, II1b, II22, II4, II6, Klf4, Mrc1, Myd88, Nlrp3, Ppary, Tgfb1, Tlr4, Tnf, Vcam1, Ccl2, Ccl5, Ccl7, Ccr2, Socs1, Socs3, Stat1, Stat3, and Stat6.

13. The method of claim 12, wherein the subject has a modulated expression or activity of one or more of MRC1, TGM2, CD23, CCL22 Relma (Fizz1, Retnla), Socs2, Irf4, Chia (Amcase), Chi3I1 (Gp39, Ykl40), Chi3l2 (Ykl39), Chi3l3(Ym1), Cxcl13, Ccl12, Ccl24, and Klf4.

14. The method of any one of the above claims, wherein the subject is not undergoing treatment with and/or is unresponsive to one or more of an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE).

15. The method of any one of the above claims, wherein the subject has evidence of AMD as confirmed by the presence of at least 1 druse greater than about 125 μm in diameter.

16. The method of any one of the above claims, wherein the subject has no evidence of prior or active choroidal neovascularization (CNV).

17. The method of any one of the above claims, wherein the subject has one or more well-demarcated GA lesions of a total area of about 2 to about 20 mm² in one or more eye.

18. The method of any one of the above claims, wherein the subject has a best-corrected visual acuity score of greater than about 35 letters or a Snellen VA equivalent of about 20/200 or better.

19. The method of any one of the above claims, wherein the subject has a GA lesion of less than one disc area up to more than 10 disc areas.

20. The method of any one of the above claims, wherein the subject has early or late stage dry macular degeneration as evidenced by several small drusen or a few medium-sized drusen.

21. The method of any one of the above claims, wherein the subject has early or late stage dry macular degeneration as evidenced by a large number of medium-sized drusen or one or more large drusen.

22. The method of any one of the above claims, wherein the subject has early or late stage dry macular degeneration as evidenced by several large drusen and/or an extensive breakdown of cells in the macula.

23. The method of any one of the above claims, wherein the method further comprises administering an additional therapeutic agent.

24. The method of claim 23, wherein the additional therapeutic agent is selected from an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE).

25. The method of claim 24, wherein the additional therapeutic agent is selected from an anti-vascular endothelial growth factor (VEGF) agent, a modulator of the complement cascade, an angiotensin-converting enzyme (ACE) inhibitor, a peroxisome proliferator-activated receptor (PPAR)-gamma agonist, a renin inhibitor, a corticosteroid, and an agent that modulates autophagy.

26. The method of any one of the above claims, wherein the subject is a human.

27. The method of any one of the above claims, wherein the administering is effected orally or intra-vascularly.

28. The method of any one of the above claims, wherein the administering is effected intraocularly or to the ocular surface.

29. The method of any one of the above claims, wherein the treatment results in a reduction in the rate of formation, growth or expansion of patches of ocular tissue atrophy or patches of tissue loss.

30. The method of any one of the above claims, wherein the treatment comprises treating, preventing, or reducing the rate of pathogenesis of the ocular disorder.

31. A method of treating diabetic retinopathy, comprising administering to a subject in need thereof an effective amount of a compound of Formula I: 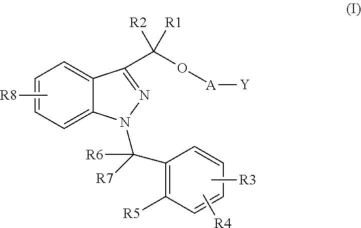
or a pharmaceutically acceptable salt thereof,
wherein
A may be a bond σ, —X₁— or —X₁—O—X₂—, in which
X₁ and X₂, which may be identical or different from each other, may be an alkyl group having from 1 to 5 carbon atoms, optionally substituted with one or more alkyl groups having from 1 to 5 carbon atoms or one or more alkoxy groups having from 1 to 3 carbon atoms,
Y is H when A is a bond σ, or Y may be H, —OH, or —N(R₁₁)(R₁₂), when A is —X₁— or —X₁—O—X₂—, in which
R₁₁ may be hydrogen, an alkyl group having from 1 to 5 carbon atoms, or R₁₁ together with R₁₂ forms a 4- to 7-membered heterocycle,
R₁₂ may be hydrogen, an alkyl group having from 1 to 5 carbon atoms, or R₁₂ together with R₁₁ forms a 4- to 7-membered heterocycle,
R₁ and R₂, which may be identical or different from each other, may be hydrogen, an alkyl group having from 1 to 5 carbon atoms,
R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,
R₅ may be hydrogen, an alkyl group having from 1 to 5 carbon atoms, an alkoxy group having from 1 to 3 carbon atoms, a halogen atom, —OH, —N(R)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms, and
R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms.

32. The method of claim 31, wherein the compound is a compound of Formula I with the proviso that when A is a σ bond, and Y, R₁, R₂, R₆, and R₇ are hydrogen atoms,
if R₈ is a hydrogen atom, then the group linked to the nitrogen atom in the 1-position of the indazole ring is different from a benzyl group, a 4-chlorobenzyl group, or a 2-4-dichlorobenzyl group,
if R₈ is a fluorine atom in the 5-position of the indazole ring, then the group linked to the nitrogen atom in the 1-position of the indazole ring is different from 5-chloro-2-methoxybenzyl group, and
if R₈ is a trifluoromethyl group in the 6-position of the indazole ring, then the group linked to the nitrogen atom in the 1-position of the indazole ring is different from a 2-4-dichlorobenzyl group.

33. A method of treating dry age-related macular degeneration (AMD) or reticular pseudodrusen disease (RPD), comprising administering to a subject in need thereof an effective amount of a compound of Formula I: 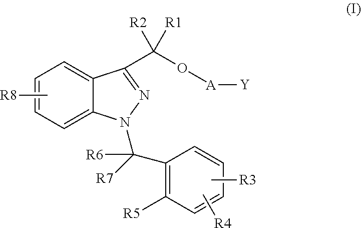
or a pharmaceutically acceptable salt thereof,
wherein
A may be a bond σ, —X₁— or —X₁—O—X₂—, in which
X₁ and X₂, which may be identical or different from each other, may be an alkyl group having from 1 to 5 carbon atoms, optionally substituted with one or more alkyl groups having from 1 to 5 carbon atoms or one or more alkoxy groups having from 1 to 3 carbon atoms,
Y is H when A is a bond σ, or Y may be H, —OH, or —N(R₁₁)(R₁₂), when A is —X₁— or —X₁—O—X₂—, in which
R₁₁ may be hydrogen, an alkyl group having from 1 to 5 carbon atoms, or R₁₁ together with R₁₂ forms a 4- to 7-membered heterocycle,
R₁₂ may be hydrogen, an alkyl group having from 1 to 5 carbon atoms, or R₁₂ together with R₁₁ forms a 4- to 7-membered heterocycle,
R₁ and R₂, which may be identical or different from each other, may be hydrogen, an alkyl group having from 1 to 5 carbon atoms,
R₃, R₄ and R₈, which may be identical or different from each other, may be hydrogen, an alkyl group having from 1 to 5 carbon atoms, an alkoxy group having from 1 to 3 carbon atoms, a halogen atom, —OH, —N(R′)(R″), —N(R′)COR″, —CN, —CONR′R″, —SO₂NR′R″, —SO₂R′, nitro and trifluoromethyl; with R′ and R″, which may be identical or different from each other, represented by hydrogen and an alkyl group having from 1 to 5 carbon atoms,
R₅ may be hydrogen, an alkyl group having from 1 to 5 carbon atoms, an alkoxy group having from 1 to 3 carbon atoms, a halogen atom, —OH, —N(R)(R″), —N(R′)COR″, nitro and trifluoromethyl, or R₅ together with one from between R₆ and R₇ forms a ring having 5 or 6 carbon atoms; with R′ and R″, which may be identical or different from each other, represented by hydrogen and an alkyl group having from 1 to 5 carbon atoms, and
R₆ and R₇, which may be identical or different from each other, may be hydrogen, an alkyl group having from 1 to 5 carbon atoms, or together form a group C═O, or one from between R₆ and R₇, together with R₅, forms a ring having 5 or 6 carbon atoms; and
a complement factor D inhibitor.

34. The method of claim 33, wherein the complement factor D inhibitor is Lampilizumab.

35. The method of claim 33 or 34, wherein the compound is a compound of Formula I with the proviso that when A is a σ bond, and Y, R₁, R₂, R₆, and R₇ are hydrogen atoms,
if R₈ is a hydrogen atom, then the group linked to the nitrogen atom in the 1-position of the indazole ring is different from a benzyl group, a 4-chlorobenzyl group, or a 2-4-dichlorobenzyl group,
if R₈ is a fluorine atom in the 5-position of the indazole ring, then the group linked to the nitrogen atom in the 1-position of the indazole ring is different from 5-chloro-2-methoxybenzyl group, and
if R₈ is a trifluoromethyl group in the 6-position of the indazole ring, then the group linked to the nitrogen atom in the 1-position of the indazole ring is different from a 2-4-dichlorobenzyl group.
